(19) United States
(12) Patent Application Publication
(10) Pub. No.: US 2016/0058837 A1
(43) Pub. Date: Mar. 3, 2016

(54) METHODS OF USING SECONDARY LYMPHOID ORGAN CHEMOKINE TO MODULATE PHYSIOLOGICAL PROCESSES IN MAMMALS

(71) Applicants: The Regents of the University of California, Oakland, CA (US); The Department of Veterans' Affairs, Washington, DC (US)

(72) Inventors: Steven M. Dubinett, Los Angeles, CA (US); Robert M. Strieter, Sherman Oaks, CA (US); Sherven Sharma, Culver City, CA (US); Raj Batra, Beverly Hills, CA (US)

(73) Assignees: The Regents of the University of California, Oakland, CA (US); The Department of Veterans' Affairs, Washington, DC (US)

(21) Appl. No.: 14/674,561

(22) Filed: Mar. 31, 2015

Related U.S. Application Data
(63) Continuation of application No. 10/756,101, filed on Jan. 13, 2004, now abandoned.
(60) Provisional application No. 60/439,554, filed on Jan. 13, 2003.

(51) Int. Cl. A61K 38/19; A61K 9/00; A61K 9/127

(52) U.S. Cl. CPC A61K 38/195; A61K 9/127; A61K 9/0019; A61K 48/00

(57) ABSTRACT

The invention is based on the disclosure provided herein that secondary lymphoid organ chemokine (SLC) inhibits the growth of syngeneic tumors in vivo. Thus, the invention provides a method of treating cancer in a mammal subject by administering a therapeutically effective amount of an SLC to the mammal. SLCs useful in the methods of the invention include SLC polypeptides, variants and fragments and related nucleic acids.

### RELATED APPLICATIONS

This application is a continuation application of U.S. patent application Ser. No. 10/756,101 filed Jan. 13, 2004, which claims priority under Section 119(e) from U.S. Provisional Application Ser. No. 60/439,554 filed Jan. 13, 2003, and which is a continuation-in-part of U.S. patent application Ser. No. 10/124,862 filed Apr. 18, 2002, which claims priority under Section 119(e) from U.S. Provisional Application Ser. No. 60/284,845 filed Apr. 18, 2001, the contents of each of which are incorporated herein by reference.

### STATEMENT OF GOVERNMENT SUPPORT

This invention was made with United States Government support under National Institutes of Health Grant RO1 CA71818 and National Institutes of Health Grants R01 CA78654, P01 1P50 CA90388, RO1 CA 85686, UC-TRDRP, NIH 1T32HL6699201, and VA Merit Review Funds. The United States Government may have certain rights in the invention.

### FIELD OF THE INVENTION

The present invention relates to methods of using secondary lymphoid organ chemokine to modulate mammalian physiological processes including those associated with pathological conditions such as cancer.

### BACKGROUND OF THE INVENTION

Understanding the immune mechanisms that influence oncogenesis, cancer regression, recurrence and metastasis is a crucial aspect of the development of new immunotherapies. In this context, artisans understand that a fundamental aspect of an immune response is the ability of an organism's immune cells to distinguish between self and non-self antigens. Consequently, clinically relevant models which seek to dissect immune mechanisms in cancer must take into account the fact that tumor cells share a genetic background with cells of the host immune system (i.e. are syngeneic). Unfortunately, many animal models of cancer which introduce cancer cell lines into an animal are confounded by immune responses that are influenced by differences between the genetic background of the host animal and the cancer cell lines that are being evaluated. Specifically, in cancer models in which host animals and cancer cell lines do not share an essentially identical genetic background, there are a variety of problems including those associated with “non-self” immune responses by the host's immune system that are akin to those seen in the rejection of transplanted organs between individuals. The non-self immune responses that can result from the host immune system's recognition of non-self antigens on autogeneic cancer cells (a phenomena which understandably does not occur in cancers), create an immune response to cancer cells that does not occur in human cancers. Therefore, there is an ongoing need for cancer models which faithfully mimic the development and progression of cancer so that clinically relevant analyses of immune mechanisms can be performed.

Effective immune responses to tumor cells require both APCs and lymphocyte effectors (see, e.g. Huang et al., Science, 264: 961-965, 1994). Because tumor cells often have limited expression of MHC antigens and lack costimulatory molecules, they are ineffective APCs (see, e.g. Restifo et al., J. Exp. Med., 177: 265-272, 1993). In addition, tumor cells secrete immunosuppressive mediators that contribute to evasion of host immune surveillance (see, e.g. Huang et al., Cancer Res., 58: 1208-1216, 1998; Sharma et al., J. Immunol., 163: 5020-5028, 1999; and Uzzo et al., J. Clin. Investig., 104: 769-776, 1999). To circumvent this problem, investigators are using ex vivo generated DCs to stimulate antitumor immune responses in vivo. In experimental murine models, DCs pulsed with tumor-associated antigenic peptides (Nair et al., Eur. J. Immunol., 27: 589-597, 1997) or transfected with tumor RNA have been shown to induce antigen-specific antitumor responses in vivo (Boczkowski et al., J. Exp. Med., 184:465-472, 1996). Similarly, fusion of DCs with tumor cells or intratumoral injection of cytokine-modified DCs has also been shown to enhance antitumor immunity (Gong et al., Nat. Med., 3: 558-561, 1997; Celluzzi et al., J. Immunol., 160: 3081-3085, 1998; Miller et al., Hum. Gene Ther., 11:53-65, 2000). Consequently, it has been suggested that effective anticancer immunity may be achieved by recruiting professional host APCs for tumor antigen presentation to promote specific T-cell activation (Soto et al., Annu. Rev. Immunol., 15: 675-705, 1997). Thus, chemokines that attract both DCs and lymphocyte effectors to lymph nodes and tumor sites could serve as potent agents in cancer immunotherapy.

Chemokines, a group of homologous, yet functionally divergent proteins, directly mediate leukocyte migration and activation and playa role in regulating angiogenesis (Baggiolini et al., Rev. Immunol., 15: 675-705, 1997). Chemokines also function in maintaining immune homeostasis and secondary lymphoid organ architecture (Jung et al., Curr. Opin. Immunol., 11: 319-325, 1999). Several chemokines are known to have antitumor activity. Tumor rejection has been noted in various murine tumor models in which tumor cells have been modified with chemokines including MIP1α, RANTES, lymphotactin, TCA3, JE/MCP-1/MCAF, MIP3α, MIP3β, and IP-10 (Luster et al., J. Exp. Med., 178: 1057-1065, 1993; Bottazzi et al., J. Immunol., 148: 1280-1285, 1992; Kellermann et al., J. Immunol., 162: 3859-3864, 1999; Sallusto et al., Eur. J. Immunol., 28: 2760-2769, 1998; Sozzani et al., J. Immunol., 161: 1083-1086, 1998; Dieu et al., J. Exp. Med., 188: 373-386, 1998; Campell et al., J. Cell Biol., 141: 1053-1059, 1998; Saeki et al., J. Immunol., 162: 2472-2475, 1999; Nagira et al., Eur. J. Immunol., 28: 1516-1523, 1998).

Secondary lymphoid tissue chemokine (SLC, also referred to as Exodus 2 or 6Ckine) is a high endothelial-derived CC chemokine normally expressed in high endothelial venules and in T-cell zones of spleen and lymph node, that strongly attracts naive T cells and DCs (Cyster et al., J. Exp. Med., 189: 447-450, 1999.24; Ogata et al., Blood, 93: 3225-3232, 1999; Chan et al., Blood, 93: 3610-3616, 1999; Hedrick et al., J. Immunol., 159: 1589-1593, 1997; Hromas et al., J. Immunol., 159: 2554-2558, 1997; Nagira et al., J. Biol. Chem., 272: 19518-19524, 1997; Tanabe et al., J. Immunol., 159: 5671-5679, 1997; Willimann et al., Eur. J. Immunol., 28: 2025-2034, 1998). SLC mediates its effects through two specific G protein-coupled seven-transmembrane domain chemokine receptors, CCR7 and CXCR3 (Yoshida et al., J. Biol. Chem. 273:7118; Jenh et al., J. Immunol. 162:3765). Whereas CCR7 is expressed on naive T cells and mature DC, CXCR3 is expressed preferentially on Th1 cytokine-producing lymphocytes with memory phenotype (Yoshida et al., J. Biol. Chem. 273:7118; Jenh et al., J. Immunol. 162:3765).

The capacity of SLC to chemoattract DCs (Kellermann et al., J. Immunol., 162: 3859-3864, 1999) is a property shared with other chemokines (Sallusto et al., Eur. J. Immunol., 28: 2760-2769, 1998; Sozzani et al., J. Immunol., 161: 1083-1086, 1998; Dieu et al., J. Exp. Med., 188: 373-386, 1998). However, SLC may be distinctly advantageous because of its capacity to elicit a Type 1 cytokine response in vivo (Sharma et al., J. Immunol., 164: 4558-4563, 2000). DCs are uniquely potent APCs involved in the initiation of immune responses (Banchereau et al., Nature (Lond.), 392: 245-252, 1998). Serving as immune system sentinels, DCs are responsible for Ag acquisition in the periphery and subsequent transport to T-cell areas in lymphoid organs where they prime specific immune responses. SLC recruits both naive lymphocytes and antigen stimulated DCs into T-cell zones of secondary lymphoid organs, colocalizing these early immune response constituents and culminating in cognate T-cell activation (Cyster et al., J. Exp. Med., 189: 447-450, 1999.24).

There is a need in the art for cancer models that faithfully mimic immune mechanisms in cancer in order to examine, for example how host cytokine profiles are modulated by SLC as well as the capacity of SLC to orchestrate effective cell-mediated immune responses to syngeneic cancer cells. In addition, there is a need for new assays of immune function as well as immunotherapeutic modalities based on such clinically relevant models. The disclosure provided herein meets these needs.

### SUMMARY OF THE INVENTION

The invention disclosed herein provides animal models which faithfully mimic immune mechanisms in cancer by utilizing host animals and cancer cells that have an essentially identical genetic background. These models are used to demonstrate the capacity of SLC to orchestrate effective cell-mediated immune responses to syngeneic cancer cells. In addition, these models can be used to evaluate host cytokine profiles that are associated with SLC modulated immune responses to syngeneic cancer cells.

As disclosed herein, the antitumor efficiency of secondary lymphoid organ chemokine was evaluated in a number of syngeneic models including transgenic mice that spontaneously develop tumors. In these transgenic mice, bilateral multifocal pulmonary adenocarcinomas develop in an organ-specific manner. As compared with compared with allogeneic models known in the art, the spontaneous tumors that arise in this transgenic mouse model do not expresses non-self antigens and therefore resemble human cancers.

In the syngeneic models disclosed herein, injection of recombinant SLC intratumorally and/or in the axillary lymph node region led to a marked reduction in tumor burden with extensive lymphocytic and DC infiltration of the tumors and enhanced survival. SLC injection in these syngeneic murine models led to significant increases in CD4 and CD8 lymphocytes as well as DC at the tumor sites, lymph nodes, and spleen. The cellular infiltrates were accompanied by the enhanced elaboration of Type 1 cytokines and the antiangiogenic chemokines IFN-γ inducible protein 10, and monokine induced by IFN-γ (MIG). In contrast, lymph node and tumor site production of the immunosuppressive cytokine transforming growth factor β was decreased in response to SLC treatment. In vitro, after stimulation with irradiated autologous tumor, splenocytes from SLC-treated mice secreted significantly more IFN-γ and granulocyte macrophage colony-stimulating factor, but reduced levels of interleukin 10. Significant reduction in tumor burden in a model in which tumors develop in an organ-specific manner provides methods for the use of SLC in the regulation of tumor immunity and cancer immunotherapy.

The invention disclosed herein has a number of embodiments. A typical embodiment of the invention is a method of inhibiting the growth of a spontaneous cancer in a mammal by administering to the mammal an amount of secondary lymphoid tissue chemokine (SLC) polypeptide sufficient to inhibit the growth of the cancer cells. In preferred methods the SLC has the polypeptide sequence shown in SEQ ID NO: 1. In these methods SLC polypeptide is typically administered to a mammal sytemically, via intratumoral injection or via intra-lymph node injection. In yet another mode of administration, an expression vector having a polynucleotide encoding a SLC polypeptide is administered to the mammal and the SLC polypeptide is produced by a mammalian cell transduced with the SLC expression vector.

A related embodiment of the invention is a method of inhibiting the growth of syngeneic cancer cells (most preferably spontaneous cancer cells) in a mammal comprising administering secondary lymphoid tissue chemokine (SLC) to the mammal; wherein the SLC is administered to the mammal by transducing the cells of the mammal with a polynucleotide encoding the SLC shown in SEQ ID NO: 1 such that the transduced cells express the SLC polypeptide in an amount sufficient to inhibit the growth of the cancer cells. Preferably the vector is administered to a mammal systemically, via intratumoral injection or via intra-lymph node injection.

Another embodiment of the invention is a method of effecting or modulating cytokine expression (e.g. changing an existing cytokine profile) in a mammal or in a population of cells derived from a mammal by exposing the population of cells to an amount of secondary lymphoid tissue chemokine (SLC) polypeptide sufficient to inhibit the growth of syngeneic tumor cells. As disclosed herein, because the syngeneic models disclosed herein demonstrate how the addition of SLC coordinately modulates cytokine expression and inhibits the growth of the tumor cells, observations of these phenomena (modulation of cytokine expression and inhibition of tumor growth) can be used in cell based assays designed to assess the effects of potential immunostimulatory or immunoinhibitory test compounds.

Another embodiment of the invention is a method of effecting an increase in the expression of Interferon-γ (IFN-γ) polypeptide and a decrease in the expression of Transforming Growth Factor-β (TGF-β) polypeptide in a population of syngeneic mammalian cells including CD8 positive T cells, CD4 positive T cells, Antigen Presenting Cells and tumor cells by exposing the population of cells to an amount of secondary lymphoid tissue chemokine (SLC) polypeptide sufficient to inhibit the growth of the tumor cells. In preferred methods, the increase in the expression of Interferon-γ (IFN-γ) polypeptides is at least about two-fold and a decrease in the expression of Transforming Growth Factor-β (TGF-β) polypeptides is at least about two-fold as measured by an enzyme linked immunoadsorbent (ELISA) assay.

### BRIEF DESCRIPTION OF THE FIGURES

FIG. 1. SLC mediates antitumor responses in immune competent mice: requirement for CD4 and CD8 lymphocyte subsets. 3LL (H-2d) or L1C2 (H-2b) cells (10⁵) were inoculated s.c. into the right supra scapular area in C57BL/6 and BALB/c mice. Five days after tumor establishment, 0.5 μg of murine recombinant SLC per injection or PBS diluent (1×) was administered three times per week intratumorally. Equivalent amounts of murine serum albumin was used as an irrelevant protein for control injections, and it did not alter the tumor volumes. Tumor volume was monitored three times per week (n=10-12 mice/group). Intratumoral SLC administration led to significant reduction in tumor volumes compared with untreated tumor-bearing mice (p<0.01). In the SLC treatment group, 40% of mice showed complete tumor eradication (A and D). SLC-mediated antitumor responses are lymphocyte dependent as evidenced by the fact that this therapy did not alter tumor growth in SCID mice (FIG. 1E). Studies performed in CD4 and CD8 knockout mice also showed a requirement for both CD4 and CD8 effector subsets for SLC-mediated tumor regression (FIG. 1, B and C).

FIG. 2. Intratumoral SLC administration augments the cytolytic capacity of lymph node (LN)-derived lymphocytes. The cytolytic capacity of lymph node-derived lymphocytes from SLC-treated and diluent control tumor-bearing mice was determined after 1 week of stimulation with irradiated 3LL tumors. Lymph node-derived lymphocytes (5×10⁶ cells/ml) were cultured with irradiated 3LL (10⁵ cells/ml) tumors at a ratio of 50:1 in a total volume of 5 ml. After a 5-day culture, the lymph node-derived lymphocytes cytolytic capacity was assessed against ⁵¹Cr-labeled 3LL tumor targets. After intratumoral SLC administration, the cytolytic capacity of LNDL was significantly enhanced above that of lymphocytes from diluent-treated tumor-bearing mice. *, p<0.01.

FIGS. 3A-3E. SLC mediates potent antitumor responses in a murine model of spontaneous lung cancer. The antitumor efficacy of SLC was evaluated in the spontaneous bronchogenic carcinoma model in transgenic mice in which the SV40 large T Ag is expressed under control of the murine Clara cell-specific promoter, CC-10 (Gabrilovich et al., Blood, 92: 4150-4166, 1998). Mice expressing the transgene develop diffuse bilateral bronchoalveolar carcinoma and have an average lifespan of 4 months. SLC (0.5 μg/injection) or the same concentration of murine serum albumin was injected in the axillary lymph node region of 4-week-old transgenic mice three times a week for 8 weeks. At 4 months when the control mice started to succumb because of progressive lung tumor growth, mice in all of the treatment groups were sacrificed, and their lungs were isolated and embedded in paraffin. H&E staining of paraffin-embedded lung tumor sections from control-treated mice evidenced large tumor masses throughout both lungs without detectable lymphocytic infiltration (3A and 3C). In contrast, the SLC therapy group evidenced extensive lymphocytic infiltration with marked reduction in tumor burden (3B and 3D). Arrows in 3D depict tumor (*1) and infiltrate (*2). (3A and 3B, ×32; 3C and 3D, ×320) 3E, reduced tumor burden in SLC-treated mice. Tumor burden was quantified within the lung by microscopy of H&E-stained paraffin-embedded sections with a calibrated graticule (a 1-cm² grid subdivided into 100 1-mm² squares). A grid square with tumor occupying >50% of its area was scored as positive, and the total number of positive squares was determined. Ten separate fields from four histological sections of the lungs were examined under high-power (×20 objective). There was reduced tumor burden in SLC-treated CC-10 mice compared with the diluent-treated control group. Median survival was 18±2 weeks for control-treated mice. In contrast, mice treated with SLC had a median survival of 34±3 weeks. (P<0.001; n=10 mice/group).

FIGS. 4A-4B. Intratumoral administration of Ad-SLC reduces lung cancer growth in vivo. Mice were inoculated with 100,000 L1C2 tumor cells and after 5 days treated intratumorally once a week for three weeks with either 10⁸ pfu of Ad-CV or Ad-SLC. At this MOI, of Ad-SLC, L1C2 tumor cells transduced in vitro secreted 10 ng/ml/10⁶ cells/24 hr of SLC. The reduction in tumor volume over time is shown in graphic form in FIG. 4A and the number of mice with complete tumor eradication after therapy is shown in table form in FIG. 4B.

FIGS. 5A and 5B show Tables 1A and 1B respectively. Table 1A shows Intratumoral SLC administration promotes Th1 cytokine and antiangiogenic chemokine release and a decline in immunosuppressive mediators. Cytokine profiles in tumors were determined in mice treated intratumorally with SLC and compared with those in diluent-treated control mice bearing tumors. Non-necrotic tumors were harvested, cut into small pieces, and passed through a sieve. Tumors were evaluated for the presence of IL-10, IL-12, GM-CSF, IFN-γ, TGF-β, VEGF, MIG, and IP-10 by ELISA and for PGE₂ by EIA in the supernatants after overnight culture. Cytokine, PGE₂, and VEGF determinations from the tumors were corrected for total protein by Bradford assay. Results are expressed as picograms per milligram total protein/24 h. Compared with tumor nodules from diluent-treated tumor-bearing controls, mice treated intratumorally with SLC had significant reductions of PGE₂, VEGF, IL-10, and TGF-β but an increase in IFN-γ, GM-CSF, IL-12, MIG, and IP-10. Experiments were repeated twice. Table 1B shows how SLC treatment of CC-10 Tag mice promotes Type 1 cytokine and antiangiogenic chemokine release and a decline in the immunosuppressive and angiogenic cytokines TGF-β and VEGF. Following axillary lymph node region injection of SLC, pulmonary, lymph node, and spleen cytokine profiles in CC-10 Tag mice were determined and compared with those in diluent-treated tumor bearing control mice and nontumor bearing syngeneic controls. Lungs were harvested, cut into small pieces, passed through a sieve, and cultured for 24 h. Splenocytes and lymph node-derived lymphocytes (5×10⁶ cells/ml) were cultured for 24 h. After culture, supernatants were harvested, cytokines quantified by ELISA, and PGE-2 determined by EIA. All determinations from lung were corrected for total protein by Bradford assay, and results are expressed in pg/milligram total protein/24 h. Cytokine and PGE-2 determinations from the spleen and lymph nodes are expressed in pg/ml. Compared with lungs from diluent-treated CC-10 tumor-bearing mice, CC-10 mice treated with SLC had significant reductions in VEGF and TGF-β but a significant increase in IFN-γ, IP-10, IL-12, MIG, and GM-CSF. Compared with diluent-treated CC-10 Tag mice, splenocytes from SLC-treated CC-10 mice had reduced levels of IFN-γ, IP-10, MIG, and IL-12 but decreased TGF-β levels as compared with diluent-treated CC-10 mice. Values given reflect mean±SE for six mice/group.

FIGS. 6A and 6B show Tables 2A and 2B respectively. Table 2A shows that SLC increases the frequency of CD4 and CD8 lymphocyte subsets secreting IFN-γ and GM-CSF and CD11c+DEC205-expressing DC. Single-cell suspensions of tumor nodules and lymph nodes from SLC and diluent-treated tumor-bearing mice were prepared. Intracytoplasmic staining for GM-CSF and IFN-γ and cell surface staining for CD4 and CD8 T lymphocytes were evaluated by flow cytometry. DC that stained positively for cell surface markers CD11c and DEC205 in lymph node and tumor nodule single-cell suspensions were also evaluated. Cells were identified as lymphocytes or DC by gating based on the forward and side scatter profiles: 15,000 gated events were collected and analyzed using Cell Quest software. Within the gated T lymphocyte population, intratumoral injection of SLC led to an increase in the frequency of CD4 and CD8 cells secreting GM-CSF and IFN-γ in the tumor nodules and lymph nodes compared with those of diluent-treated tumor-bearing control mice. Within the gated DC population, there was a significant increase in the frequency of DC in the SLC-treated tumor-bearing mice compared with the diluent-treated control tumor-bearing mice. For DC staining, MCF is for DEC205. MCF, mean channel fluorescence. Experiments were repeated twice. Table 2B shows that SLC treatment of CC-10 Tag mice leads to enhanced dendritic and T cell infiltrations of tumor sites, lymph nodes and spleen. Single-cell suspensions of tumor nodules, lymph nodes, and spleens from SLC and diluent-treated tumor-bearing mice were prepared. Intracytoplasmic staining for GM-CSF and IFN-γ and cell surface staining for CD4 and CD8 T lymphocytes were evaluated by flow cytometry. DCs that stained positive for cell surface markers CD11c and DEC205 in lymph node, tumor nodule, and spleen single-cell suspensions were also evaluated. Cells were identified as lymphocytes or DCs by gating based on the forward and side scatter profiles; 15,000 gated events were collected and analyzed using Cell Quest software. Within the gated T-lymphocyte population from mice treated with SLC, there was an increase in the frequency of CD4+ and CD8+ cells secreting GM-CSF and IFN-γ in the tumor sites, lymph nodes, and spleens compared with those of diluent-treated tumor-bearing control mice. Within the gated DC population, there was a significant increase in the frequency of DCs in the SLC-treated tumor-bearing mice compared with the diluent-treated control tumor-bearing mice.

FIGS. 7A and 7B show Tables 3A and 3B respectively. FIG. 3A shows the specific systemic induction of type 1 cytokines and down-regulation of IL-10 after SLC treatment. Splenic or lymph node-derived lymphocytes (5×10⁶ cells/ml) were cultured with irradiated 3LL (10⁵ cells/ml) tumors at a ratio of 50:1 in a total volume of 5 ml. After overnight culture, supernatants were harvested, and GM-CSF, IFN-γ, IL-12, and IL10 were determined by ELISA. After stimulation with irradiated tumor cells, splenocytes and lymph node-derived cells from SLC-treated mice secreted significantly enhanced levels of IFN-γ, GM-CSF, and IL-12 but reduced levels of IL-10 compared with diluent-treated bearing mice. Results are expressed as picograms per milliliter. Experiments were repeated twice. Table 3B shows the systemic induction of type 1 cytokines and downregulation of IL-10 after SLC treatment. Splenic lymphocytes (5×10⁶ cells/ml) were cultured with irradiated CC-10 (10⁵ cells/ml) tumors at a ratio of 50:1 in a total volume of 5 ml. After overnight culture, supernatants were harvested and GM-CSF, IFN-γ, and IL-10 were determined by ELISA. After stimulation with irradiated tumor cells, splenocytes secreted significantly more IFN-γ and GM-SCF but reduced levels of IL-10 from SLC-treated mice compared to diluent-treated tumor-bearing mice. Results are expressed in pg/ml (^{α}P<0.01 compared with diluent-treated mice as well as SLC-treated constitutive levels). Values given reflect mean±SE for five mice/group.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art, such as, for example, the widely utilized molecular cloning methodologies described in see Ausubel et al., Current Protocols in Molecular Biology, Wiley Interscience Publishers, (1995) and Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

Abbreviations used herein include: APC, antigen-presenting cell; SLC, secondary lymphoid organ chemokine; DC, dendritic cell; IP-10, IFN-γ inducible protein 10; TGF-β, transforming growth factor β; GM-CSF, granulocyte macrophage colony-stimulating factor; IL, interleukin; FBS, fetal bovine serum; mAb, monoclonal antibody; VEGF, vascular endothelial growth factor; EIA, enzyme immunoassay; SV40 TAg, simian virus 40 large T antigen; Ag, antigen; PGE2, prostaglandin E2; PE, phycoerythrin; LN, lymph node.

### A. Brief Characterization of Features of the Invention

The invention is based on the discoveries disclosed herein that Secondary Lymphoid-Tissue Chemokine (SLC) modulates cytokine profiles in an immune response to syngeneic tumor cells and can inhibit the growth of these cells. The disclosure provided herein demonstrates the antitumor efficiency of SLC in a clinically relevant mouse model where the mice spontaneously develop tumors. For example, injection of recombinant SLC (e.g. in the axillary lymph node region) leads to a marked reduction in this syngeneic tumor burden with extensive lymphocytic and DC infiltration of the tumors and enhanced survival. SLC injection led to significant increases in CD4 and CD8 lymphocytes as well as DC at the tumor sites, lymph nodes, and spleen.

As discussed below, the cellular infiltrates observed at the site of the syngeneic tumors were accompanied by the enhanced elaboration of Type 1 cytokines and the antiangiogenic chemokines IFN-γ inducible protein 10, and monokine induced by IFN-γ (MIG). In contrast, lymph node and tumor site production of the immunosuppressive cytokine transforming growth factor 13 was decreased in response to SLC treatment. In vitro, after stimulation with irradiated autologous tumor, splenocytes from SLC-treated mice secreted significantly more IFN-γ and granulocyte macrophage colony-stimulating factor, but reduced levels of interleukin 10. Significant reduction in tumor burden in a model in which tumors develop in an organ-specific manner provides a strong rationale for additional evaluation of SLC in regulation of tumor immunity and its use in lung cancer immunotherapy.

In view of the disclosure provided herein and because DCs are potent APCs that function as principle activators of T cells, the capacity of SLC to facilitate the colocalization of both DC and T cells is shown to reverse tumor-mediated immune suppression and orchestrate effective cell mediated immune responses in a syngeneic context. In addition to its immunotherapeutic potential, SLC has been found to have potent angiostatic effects (Soto et al., Annu. Rev. Immunol., 15: 675-705, 1997), thus adding additional support for its use in cancer therapy.

Using transplantable murine lung cancer models, we show that the antitumor efficacy of SLC is T cell-dependent. In these transplant models, the antitumor efficacy of SLC was determined using transplantable tumors propagated at s.c. sites. In the transplantable models, recombinant SLC administered intratumorally led to complete tumor eradication in 40% of the treated mice. The SLC-mediated antitumor response was dependent on both CD4 and CD8 lymphocyte subsets and was accompanied by DC infiltration of the tumor. In recent studies that directly support the antiangiogenic capacity of this chemokine, Arenberg et al. (Arenberg et al., Cancer Immunol. Immunother., 49:587-592, 2000) have reported that SLC inhibits human lung cancer growth and angiogenesis in a SCID mouse model.

The spontaneous tumor model discussed herein demonstrates the antitumor properties of SLC in a clinically relevant model of cancer in which adenocarcinomas develop in an organ-specific manner. Specifically, in this model, transgenic mice expressing SV40 large TAg transgene under the control of the murine Clara cell-specific promoter, CC-10, develop diffuse bilateral bronchoalveolar carcinoma and have an average lifespan of 4 months (Magdaleno et al., Cell Growth Differ., 8: 145-155, 1997). The antitumor activity of SLC is determined in the spontaneous model for lung cancer by injecting recombinant SLC into the axillary lymph node region of the transgenic mice. The rationale for injecting SLC in the lymph node region was to colocalize DC to T-cell areas in the lymph nodes where they can prime specific antitumor immune responses. In many clinical situations access to lymph node sites for injection may also be more readily achievable than intratumoral administration. These results show that this approach is effective in generating systemic antitumor responses. SLC injected in the axillary lymph node regions of the CC-10 TAg mice evidenced potent antitumor responses with reduced tumor burden and a survival benefit as compared with CC-10 TAg mice receiving diluent control injections. The reduced tumor burden in SLC-treated mice was accompanied by extensive lymphocytic as well as DC infiltrates of the tumor sites, lymph nodes, and spleens.

The cytokine production from tumor sites, lymph nodes, and spleens of the CC-10 TAg mice was also altered as a result of SLC therapy. The following cytokines were measured: VEGF, IL-10, PGE-2, TGF-β, IFN-γ, GMCSF, IL-12, MIG, and IP-10 (Table 1B). The production of these cytokines was evaluated for the following reasons: the tumor site has been documented to be an abundant source of PGE-2, VEGF, IL-10, and TGF-β, and the presence of these molecules at the tumor site has been shown to suppress immune responses (Huang et al., Cancer Res., 58: 1208-1216, 1998; Gabrilovich et al., Nat. Med., 2: 1096-1103, 1996; Bellone et al., Am. J. Pathol., 155: 537-547, 1999). VEGF, PGE-2, and TGF-β have also been documented previously to promote angiogenesis (Fajardo et al., Lab. Investig., 74: 600-608, 1996; Ferrara, N. Breast Cancer Res. Treat., 36: 127-137, 1995; Tsujii et al., Cell, 93: 705-716, 1998). Antibodies to VEGF, TGF-β, PGE-2, and IL-10 have the capacity to suppress tumor growth in in vivo model systems. VEGF has also been shown to interfere with DC maturation (Gabrilovich et al., Nat. Med., 2: 1096-1103, 1996). Both IL-10 and TGF-β are immune inhibitory cytokines that may potently suppress Ag presentation and antagonize CTL generation and macrophage activation (Sharma et al., J. Immunol., 163: 5020-5028, 1999; Bellone et al., Am. J. Pathol., 155: 537-547, 1999). Although at higher pharmacological concentrations IL-10 may cause tumor reduction, physiological concentrations of this cytokine suppress antitumor responses (Sharma et al., J. Immunol., 163: 5020-5028, 1999; Sun et al., Int. J. Cancer, 80: 624-629, 1999; Halak et al., Cancer Res., 59: 911-917, 1999; Stolina et al., J. Immunol., 164: 361-370, 2000). Before SLC treatment in the transgenic tumor bearing mice, the levels of the immunosuppressive proteins VEGF, PGE-2, and TGF-β were elevated when compared with the levels in normal control mice. There was no such increase with IL-10. Similarly there were not significant alterations in IL-4 and IL-5 after SLC therapy. SLC-treated CC-10 TAg mice showed significant reductions in VEGF and TGF-β. The decrease in immunosuppressive cytokines was not limited to the lung but was evident systemically. SLC treatment of CC-10 TAg transgenic mice led to a decrease in TGF-β in lymph node-derived cells and reduced levels of PGE-2 and VEGF from splenocytes. Thus, benefits of a SLC-mediated decrease in these cytokines include promotion of antigen presentation and CTL generation (Sharma et al., J. Immunol., 163: 5020-5028, 1999; Bellone et al., Am. J. Pathol., 155: 537-547, 1999), as well as a limitation of angiogenesis (Fajardo et al., Lab. Investig., 74: 600-608, 1996; Ferrara, N. Breast Cancer Res. Treat., 36: 127-137, 1995; Tsujii et al., Cell, 93: 705-716, 1998).

It is well documented that successful immunotherapy shifts tumor specific T-cell responses from a type 2 to a type 1 cytokine profile (Hu et al., J. Immunol., 161: 3033-3041, 1998). Responses depend on IL-12 and IFN-γ to mediate a range of biological effects, which facilitate anticancer immunity. IL-12, a cytokine produced by macrophages (Trinchieri et al., 70: 83-243, 1998) and DC (Johnson et al., J. Exp. Med., 186:1799-1802, 1997), plays a key role in the induction of cellular immune responses (Ma et al., Chem. Immunol., 68: 1, 1997). IL-12 has been found to mediate potent antitumor effects that are the result of several actions involving the induction of CTL, Type 1-mediated immune responses, and natural killer activation (Trinchieri et al., 70: 83-243, 1998), as well as the impairment of tumor vascularization (Voest et al., J. Natl. Cancer Inst., 87: 581-586, 1995). IP-10 and MIG are CXC chemokines that chemoattract activated T cells expressing the CXCR3 chemokine receptor (Loetscher et al., J. Exp. Med., 184:963-969, 1996). Both IP-10 and MIG are known to have potent antitumor and antiangiogenic properties (Luster et al., J. Exp. Med., 178: 1057-1065, 1993; Brunda et al., J. Exp. Med., 178: 1223-1230, 1993; Arenberg et al., J. Exp. Med., 184: 981-992, 1996; Sgadari et al., Blood, 89: 2635-2643, 1997). The lungs of SLC treated CC-10 TAg mice revealed significant increases in IFN-γ, IL-12, IP-10, MIG, and GM-CSF. MIG and IP-10 are potent angiostatic factors that are induced by IFN-γ (Arenberg et al., J. Exp. Med., 184: 981-992, 1996; Strieter et al., Biochem. Biophys. Res. Commun., 210: 51-57, 1995; Tannenbaum et al., J. Immunol., 161: 927-932, 1998) and may be responsible in part for the tumor reduction in CC-10 TAg mice after SLC administration. Because SLC is documented to have direct antiangiogenic effects (Soto et al., Annu. Rev. Immunol., 15: 675-705, 1997; Arenberg et al., Am. J. Resp. Crit. Care Med., 159:A746, 1999), the tumor reductions observed in this model maybe attributable to T cell-dependent immunity as well as participation by T cells secreting IFN-γ in inhibiting angiogenesis (Tannenbaum et al., J. Immunol., 161: 927-932, 1998). Hence, an increase in IFN-γ at the tumor site of SLC-treated mice would explain the relative increases in IP-10 and MIG. Both MIG and IP-10 are chemotactic for stimulated CXCR3-expressing T lymphocytes that could additionally amplify IFN-γ at the tumor site (Farber et al., J. Leukoc. Biol., 61: 246-257, 1997). Flow cytometric determinations revealed that both CD4 and CD8 cells were responsible for the increased secretion of GM-CSF and IFN-γ in SLC-treated mice. An increase in GM-CSF in SLC-treated mice could enhance DC maturation and antigen presentation (Banchereau et al., Nature (Lond.), 392: 245-252, 1998). Additional studies are necessary to precisely define the host cytokines that are critical to the SLC-mediated antitumor response.

The increase in the Type 1 cytokines was not limited to the lung but was evident systemically. SLC treatment of CC-10 TAg transgenic mice led to systemic increases in Type I cytokines and antiangiogenic chemokines. Hence, splenocytes from SLC-treated CC-10 TAg mice had an increase in GM-CSF, IL-12, MIG, and IP-10 as compared with diluent-treated CC-10 TAg mice. Similarly, lymph node-derived cells from SLC-treated mice secreted significantly enhanced levels of IFN-γ, IP-10, MIG, and IL-12. Recent studies suggest that the evaluation of type 1 responses at the LN sites may provide insights into antitumor responses in patients receiving immune therapy (Chu et al., Eur. J. Nuc. Med., 26: s50-53, 1999). The increase in GM-CSF and IFN-γ in the spleen and lymph nodes of SLC-treated mice could in part be explained by an increase in the frequency of CD4 and CD8 cells secreting these cytokines. The increase in Type 1 cytokines was in part attributable to an increase in specificity against the autologous tumor; when cocultured with irradiated CC-10 TAg tumor cells, splenocytes from SLC-treated CC-10 TAg mice secreted significantly increased amounts of GM-CSF and IFN-γ but reduced levels of IL-10. Cell surface staining of CC-10 cells followed by flow cytometry did not show detectable levels of MHC class II molecules. Although the tumor did not show MHC class II expression, CD4+ type 1 cytokine production may have occurred because splenic APC were present in the assay. Although in vitro tumor-stimulated splenic T cells from SLC-treated mice showed reduced expression of IL-10, SLC therapy did not lead to a decrease of IL-10 levels in vivo. The in situ microenvironment may provide other important factors from cellular constituents in addition to T cells that determines the overall levels of IL-10.

Taken together, the disclosure provided herein demonstrates how the administration of SLC, for example SLC injected in the axillary lymph node region in a clinically relevant spontaneous lung cancer model leads to the generation of systemic antitumor responses. Without being bound by a specific theory, the antitumor properties of SLC may be attributable to its chemotactic capacity in colocalization of DCs and T cells, as well as the induction of key cytokines such as IFN-γ, IP-10, MIG, and IL-12. Using the models disclosed herein, additional studies can delineate the importance of each of these cytokines in SLC-mediated antitumor responses. The potent antitumor properties demonstrated in this model of spontaneous bronchoalveolar carcinoma provide a strong rationale for additional evaluation of SLC regulation of tumor immunity and its use in immunotherapy for cancers such as cancers of the lung.

As described in detail below, the invention described herein has a number of embodiments. Typical embodiments include methods of modulating syngeneic physiological processes in mammals, for example effecting an increase in the expression of soluble cytokines such as Interferon-γ (IFN-γ) polypeptides and a decrease in the expression of soluble cytokines such as Transforming Growth Factor-β (TGF-β) polypeptides in a population of syngeneic mammalian cells including CD8 positive T cells, CD4 positive T cells, Antigen Presenting Cells and tumor cells by exposing the population of cells to an amount of secondary lymphoid tissue chemokine (SLC) polypeptide sufficient to inhibit the growth of the tumor cells. A closely related embodiment is a method of treating cancer or hyperproliferative cell growth in a mammal by administering a therapeutically effective amount of an SLC to the mammal.

“Mammal” for purposes of treatment or therapy refers to any animal classified as a mammal, including humans, domestic and farm animals, and zoo, sports, or pet animals, such as dogs, horses, cats, cows, etc. Preferably, the mammal is human.

The terms “cancer”, “cancerous”, or “malignant” refer to or describe the physiological condition in mammals that is typically characterized by unregulated cell growth. Examples of cancer include but are not limited to, carcinoma, lymphoma, blastoma, sarcoma, and leukemia. More particular examples of such cancers include adenocarcinoma, breast cancer, ovarian cancer, colon cancer, colorectal cancer, rectal cancer, squamous cell cancer, small-cell lung cancer, non-small cell lung cancer, Hodgkin's and non-Hodgkin's lymphoma, testicular cancer, esophageal cancer, gastrointestinal cancer, renal cancer, pancreatic cancer, glioblastoma, cervical cancer, glioma, liver cancer, bladder cancer, hepatoma, endometrial carcinoma, salivary gland carcinoma, kidney cancer, liver cancer, prostate cancer, vulval cancer, thyroid cancer, hepatic carcinoma and various types of head and neck cancer.

One of the focal issues in designing active cancer immunotherapy is that cancer cells are derived from normal host cells. Thus, the antigenic profile of cancer cells closely mimics that of normal cells. In addition, tumor antigens are not truly foreign and tumor antigens fit more with a self/altered self paradigm, compared to a non-self paradigm for antigens recognized in infectious diseases and organ transplants (see, e.g. Lewis et al., Semin Cancer Biol 6(6): 321-327 (1995)). In this context, an important aspect of the present invention is the characterization of the effects of SLC in an animal model where the cancer cells are spontaneous and the immune cells which respond to the cancer cells are therefore syngeneic. In this context, syngeneic is known in the art to refer to an extremely close genetic similarity or identity especially with respect to antigens or immunological reactions. Syngeneic systems include for example, models in which organs and cells (e.g. cancer cells and their non-cancerous counterparts) come from the same individual, and/or models in which the organs and cells come from different individual animals that are of the same inbred strain. Syngeneic models are particularly useful for studying oncogenesis and chemotherapeutic molecules. A specific example of a syngeneic model is the CC-10 TAg transgenic mouse model of spontaneous bronchoalveolar carcinoma described herein. In this context, artisans in the field of immunology are aware that, during mammalian development the immune system is tolerized to self antigens (e.g. those encoded by genes in the animal's germline DNA). As T-Ag is present in the germline of the transgenic animal, the transgenic animal's immune system is tolerized to this protein during maturation of the immune system.

In contrast to syngeneic, the term allogeneic is used to connote a genetic dissimilarity between tissues or cells that is sufficient to effect some type of immunological mechanism or response to the different antigens present on the respective tissues or cells. A specific example of an allogeneic model is one in which cancer cells from one strain of mice are transplanted into a different strain of mice. Allogeneic models are particularly useful for studying transplantation immunity and for the evaluation of molecules that can suppress the immune response to non-self antigens present on the transplanted tissues.

In order to provide clinically relevant paradigms for studying various pathologies which involve the immune system, animal models designed to assess immune responses must be predicated on an understanding of the immune system responds to foreign (non-self) tissues. In this context, those skilled in the field of transplantation immunity understand that an animal's immune response to allogeneic tissues is very different from an animal's immune response to syngeneic tissues (that is if a response will even occur). This is illustrated, for example by the need for immunosuppressive agents in allogeneic organ transplants (immunosuppressive agents are needed to inhibit a response to non-self antigens present on the transplanted tissues). Therefore clinically relevant models cannot mix different immunophenotypes without considering and characterizing the profound implications that this has on immune response. Because the tumor cells are syngeneic in the CC-10 TAg transgenic mouse model of spontaneous bronchoalveolar carcinoma described herein, this model specifically avoids the problems associated with a confounding immune responses that result from the mixing different immunophenotypes.

As is known in the art, cytokines are crucial mediators of immune response. In this context, different cytokines, different concentrations of cytokines and/or different combinations of cytokines are used to generate a specific immune response in a specific context. In this regard, it is known in the art that different immune responses involve different cytokine profiles. Therefore, the inherent differences an immune response to non-self tissues as compared to an immune response to self tissues result in part from inherent differences in the cytokine profiles involved in each response.

Clinically relevant paradigms for the general examination of an immune response must also take a number of other factors into account. For example it is known in the art that certain murine strains demonstrate a high variability in their immune response to identical agents. See, for example, Dreau et al., Physiolo Behav 2000 70(5): 513-520 which teaches that the murine strains C57BL6, BALB/c and BDF(1) demonstrate high variability in their immune response to 2-deoxy-D-glucose induced stress. In addition, it is known that genetic polymorphisms among common mouse strains can significantly influence early cytokine production in stimulated naïve CD4 T cells (see, e.g. Lo et al., Int Rev Immunol 1995, 13(2):147-160). Therefore, clinically relevant models of immune responsiveness should not mix tissues and cells from murine strains which are known to demonstrate high variability in their immune response without considering and characterizing the profound implications that this has on an immune response generated by model which mixes tissues and cells from different murine strains. Because there is no mixing of tissues and cells from different murine strains in the CC-10 TAg transgenic mouse model of spontaneous bronchoalveolar carcinoma described herein, this model specifically avoids the problems associated with a confounding immune responses that result from the mixing different immunophenotypes.

Clinically relevant paradigms for the specific evaluation of an immune response to cancer cells must also take a number of factors into account. For example many tumor cell lines have been selected to have certain characteristics such as enhanced invasive and metastatic behavior (see, e.g. Poste et al., Cancer Res. 42(7): 2770-2778 (1982)). As is known in the art, the selection for such characteristics can alter the factors such as the immunogenicity of such cell lines which, in turn, can confound models of immune responses that utilize such lines (see, e.g. De Baetselier et al., Nature 1980 13; 288(5787): 179-181). As is also known in the art, the growth of cell lines in tissue culture selects for an outgrowth of clones having characteristics associated with the greatest fitness in the culture medium, characteristics which are not necessarily consistent with tumor cell growth in vivo. Because the CC-10 TAg transgenic mouse model described herein produces spontaneous cancer cells (as compared to cell lines), this model specifically avoids the problems associated with the use of cell lines which have been subjected to specific (and non-specific) selective pressures during their period in tissue culture.

In addition to the above-mentioned problems with tumor cells, there are related problems associated with the use of cell lines in such models that relate to the ability of many cultured tumor lines to produce cytokines such as those that facilitate tumor growth. Specifically, it is known in the art that certain tumor cell lines express cytokines that are not produced by their non-cancerous counterparts or which are produced in quantities in normal tissues (see, e.g. Stackpole et al., In Vitro Cell Dev Biol Anim 1995, 31(3):244-251 and which discusses the autocrine growth of B16 melanoma clones and Shimizu et al., Cancer Res 1996, 56(14):3366-3370 which discusses the autocrine growth of colon carcinoma colon 26 clones). In contexts where one is evaluating an immune response or measuring a cytokine profile in an immune response, the use of cell lines in cancer model can be confounded by the presence of cytokines produced by the cell line (which can change the cytokine profile in these cells' environment). Therefore, in methods which seek to evaluate and/or modulate a cytokine profile, for example in clinically relevant models of immune responsiveness, artisans should not utilize cytokine generating cell lines into mice without considering and characterizing the profound implications that the presence of cell line produced cytokines has on an immune response generated by model.

As noted above, skilled artisans understand that the immune system responds to non-self tissues (e.g. allogeneic transplants) differently than it does to self tissues (e.g. a syngeneic transplant). As the ability to distinguish between self and non-self is a fundamental aspect of immunity, those skilled in the art understand that an immune reaction observed in response to a foreign tissue is not predictive of an immune response to a self tissue (that is if an immune response will even occur). This is illustrated, for example, by the need for individuals who have received allogeneic organ transplants to take immunosuppressive drugs. Consequently, any clinically relevant model of immune response must take this fundamental aspect of immunity into account, particularly ones designed to assess an immune response to cancer, a pathology which is characterized by the aberrant growth of self tissues. As the transgenic mouse model that is used herein does not expose the animal's immune system to non-self antigens, does not mix cells and tissue from strains of mice that have been observed to have different immunological characteristics and is instead directed to evaluating an immune response to spontaneous tumors, the data provided by this model is clinically relevant in the context of human cancers.

### B. Typical Methodologies for Practicing Embodiments of the Invention

A number of the methods disclosed herein are related to general methods known in the art that can be used to study the effects of SLC in the context of immunological responses to non-self (i.e. allogeneic) tissues such as genetically non-identical cancer cells transplanted into host animals.

The methods disclosed herein may be employed in protocols for treating pathological conditions in mammals such as cancer or immune-related diseases. In typical methods, SLC polypeptide is administered to a mammal, alone or in combination with still other therapeutic agents or techniques. Diagnosis in mammals of the various pathological conditions described herein can be made by the skilled practitioner. Diagnostic techniques are available in the art which allow, e.g., for the diagnosis or detection of cancer or immune related disease in a mammal. For instance, cancers may be identified through techniques, including but not limited to, palpation, blood analysis, x-ray, NMR and the like. For example, a wide variety of diagnostic factors that are known in the art to be associated with cancer may be utilized such as the expression of genes associated with malignancy (including PSA, PSCA, PSM and human glandular kallikrein expression) as well as gross cytological observations (see e.g. Bocking et al., Anal Quant Cytol. 6(2):74-88 (1984); Eptsein, Hum Pathol. 1995 February; 26(2):223-9 (1995); Thorson et al., Mod Pathol. 1998 June; 11(6):543-51; Baisden et al., Am J Surg Pathol. 23(8):918-24 91999)).

The methods of the invention are useful in the treatment of hyperproliferative disorders and cancers, and are particularly useful in the treatment of solid tumors. Types of solid tumors that may be treated according to the methods of the invention include, but are not limited to lung cancer, melanoma, breast cancer, tumors of the head and neck, ovarian cancer, endometrial cancer, urinary tract cancers, stomach cancer, testicular cancer, prostate cancer, bladder cancer, pancreatic cancer, leukemia, lymphoma, bone cancer, liver cancer, colon cancer, rectal cancer, metastases of the above, and metastases of unknown primary origin. For example, in preferred embodiments of the invention, SLC is administered to modulate cytokine profiles and/or inhibit the growth of spontaneous tumor cells of the adenocarcinoma lineage (as is demonstrated herein in the transgenic mouse model). As is known in the art, tumor cells of the adenocarcinoma lineage can occur spontaneously in a number of different organ systems (see, e.g., Yagi et al., Gan No Rinsho 1984 30(11):1392-1397).

Polypeptides useful in the methods of the invention encompass both naturally occurring proteins as well as variations and modified forms thereof. As noted above, “SLC polypeptide or protein” is meant a Secondary Lymphoid-Tissue Chemokine. SLC includes naturally occurring mammalian SLCs, and variants and fragments thereof, as defined below. Preferably the SLC is of human or mouse origin (see, e.g. SEQ ID NOS: 1 and 2 in Table 4 respectively). Most preferably the SLC is human SLC. Human SLC has been cloned and sequenced (see, e.g. Nagira et al. (1997) J Biol Chem 272:19518; the contents of which are incorporated by reference). Consequently the cDNA and amino acid sequences of human SLC are known in the art (see, e.g. Accession Nos. BAA21817 and AB002409). Mouse SLC has also been cloned and sequenced (see, e.g. Accession Nos. NP_{—}035465 and NM_{—}011335). Hromas et al. (1997) J. Immunol 1.59:2554; Hedrick et al. (1997) J. Immunol 159:1589; and Tanabe et al. (1997) J. Immunol 1.59:5671; the contents of which are incorporated herein by reference.

SLC polypeptides for use in the methods disclosed herein can be SLC variants, SLC fragments, analogues, and derivatives. By “analogues” is intended analogues of either SLC or an SLC fragment that comprise a native SLC sequence and structure, having one or more amino acid substitutions, insertions, or deletions. Peptides having one; or more peptoids (peptide mimics) are also encompassed by the term analogues (WO 91/04282). By “derivatives” is intended any suitable modification of SLC, SLC fragments, or their respective analogues, such as glycosylation, phosphorylation, or other addition of foreign moieties (e.g. Pegylation as described below), so long as the desired activity is retained. Methods for masking SLC fragments, analogues, and derivatives are available in the art.

In an illustrative SLC derivative, a polyol, for example, can be conjugated to an SLC molecule at one or more amino acid residues, including lysine residues, as disclosed in WO 93/00109. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to an SLC protein or variant is termed “pegylation.” However, those skilled in the art recognize that other polyols, such as, for example, poly(propylene glycol) and polyethylene-polypropylene glycol copolymers, can be employed using the techniques for conjugation described herein for PEG. The degree of pegylation of an SLC variant of the present invention can be adjusted to provide a desirably increased in vivo half-life (hereinafter “half-life”), compared to the corresponding non-pegylated protein.

A variety of methods for pegylating proteins have been described. See, e.g., U.S. Pat. No. 4,179,337 (issued to Davis et al.), disclosing the conjugation of a number of hormones and enzymes to PEG and polypropylene glycol to produce physiologically active non-immunogenic compositions. Generally, a PEG having at least one terminal hydroxy group is reacted with a coupling agent to form an activated PEG having a terminal reactive group. This reactive group can then react with the α- and ε-amines of proteins to form a covalent bond. Conveniently, the other end of the PEG molecule can be “blocked” with a non-reactive chemical group, such as a methoxy group, to reduce the formation of PEG-crosslinked complexes of protein molecules.

As used herein, the SLC gene and SLC protein includes the murine and human SLC genes and proteins specifically described herein, as well as biologically active structurally and/or functionally similar variants or analog of the foregoing. SLC peptide analogs generally share at least about 50%, 60%, 70%, 80%, 90% or more amino acid homology (using BLAST criteria). For example, % identity values may be generated by WU-BLAST-2 (Altschul et al., 1996, Methods in Enzymology 266:460-480; http://blast.wustl/edu/blast/README.html). SLC nucleotide analogs preferably share 50%, 60%, 70%, 80%, 90% or more nucleic acid homology (using BLAST criteria). In some embodiments, however, lower homology is preferred so as to select preferred residues in view of species-specific codon preferences and/or optimal peptide epitopes tailored to a particular target population, as is appreciated by those skilled in the art. Fusion proteins that combine parts of different SLC proteins or fragments thereof, as well as fusion proteins of a SLC protein and a heterologous polypeptide are also included. Such SLC proteins are collectively referred to as the SLC-related proteins, the proteins of the invention, or SLC.

The term “variant” refers to a molecule that exhibits a variation from a described type or norm, such as a protein that has one or more different amino acid residues in the corresponding position(s) of a specifically described protein. An analog is an example of a variant protein. As used herein, the SLC-related gene and SLC-related protein includes the SLC genes and proteins specifically described herein, as well as structurally and/or functionally similar variants or analog of the foregoing. SLC peptide analogs generally share at least about 50%, 60%, 70%, 80%, 90% or more amino acid homology (using BLAST criteria). SLC nucleotide analogs preferably share 50%, 60%, 70%, 80%, 90% or more nucleic acid homology (using BLAST criteria). In some embodiments, however, lower homology is preferred so as to select preferred residues in view of species-specific codon preferences and/or optimal peptide epitopes tailored to a particular target population, as is appreciated by those skilled in the art.

Embodiments of the invention disclosed herein include a wide variety of art-accepted variants or analogs of SLC proteins such as polypeptides having amino acid insertions, deletions and substitutions. SLC variants can be made using methods known in the art such as site-directed mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the SLC variant DNA. Resulting mutants can be tested for biological activity. Sites critical for binding can be; determined by structural analysis such as crystallization, photoaffinity labeling, or nuclear magnetic resonance. See, deVos et al. (1992) Science 255:306 and Smith et al. (1992:) J. Mol. Biol. 224:899.

As is known in the art, conservative amino acid substitutions can frequently be made in a protein without altering the functional activity of the protein. Proteins of the invention can comprise conservative substitutions. Such changes typically include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

Scanning amino acid analysis can also be employed to identify one or more amino acids along a contiguous sequence that is involved in a specific biological activity such as a protein-protein interaction. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions (Creighton, The Proteins, (W.H. Freeman & Co., N.Y.); Chothia, J. Mol. Biol., 150:1 (1976)). If alanine substitution does not yield adequate amounts of variant, an isosteric amino acid can be used.

Variant SLC proteins and SLC polypeptide fragments useful in the methods of the present invention must possess SLC biological activity. Specifically, they must possess the desired biological activity of the native protein, that is, the dendritic cell chemoattractant activity, angiostatic activity or anti-tumor activity as described herein. For the purposes of the invention, a “SLC variant” will exhibit at least 30% of a dendritic cell-chemoattractant activity, tumor inhibitory activity or angiostatic activity of the SLC. More typically, variants exhibit more than 60% of at least one of these activities; even more typically, variants exhibit more than 80% of at least one of these activities. As disclosed herein, the biological activity of a SLC protein may also be assessed by examining the ability of the SLC to modulate cytokine expression in vivo such as effecting an increase in the expression of Interferon-γ (IFN-γ) polypeptides and a decrease in the expression of Transforming Growth Factor-β (TGF-β) polypeptides in a population of syngeneic mammalian cells including CD8 positive T cells, CD4 positive T cells, Antigen Presenting Cells and tumor cells. Alternatively the biological activity of a SLC protein may also be assessed by exposing the population of cells to an amount of secondary lymphoid tissue chemokine (SLC) polypeptide and examining the ability that this molecule has to inhibit the growth of syngeneic tumor cells.

The SLC may be administered directly by introducing a SLC polypeptide, SLC variant or SLC fragment into or onto the subject. Alternatively, the SLC may be produced in situ following the administration of a polynucleotide encoding a SLC polypeptide, SLC variant or SLC fragment may be introduced into the subject.

The SLC agents of the invention comprise native SLC polypeptides, native SLC nucleic acid sequences, polypeptide and nucleic acid variants, antibodies, monoclonal antibodies, and other components that are capable of blocking the immune response through manipulation of SLC expression, activity and receptor binding. Such components include, for example, proteins or small molecules that interfere with or enhance SLC promoter activity; proteins or small molecules that attract transcription regulators; polynucleotides, proteins or small molecules that stabilize or degrade SLC mRNA; proteins or small molecules that interfere with receptor binding; and the like.

It is recognized that the invention is not bound by any particular method. Having recognized that SLC is chemotactic to mature dendritic cells, and T cells, any means of suppressing or enhancing SLC activity, for example, by interfering with receptor binding, interfering with SLC promoter activity, interfering with gene expression, mRNA stability, or protein stability, etc. can be used to modulate the primary immune response and are encompassed by the invention. The amino acid and DNA sequence of SLC are known in the art. See, for example, Pachynski et al. (1998) J. Immunol. 161:952; Yoshida et al. (1998) J. Biol. Chem. 273:7118, Nagira et al. (1998) Eur. J. Immunol. 28:1516-1523; Nagira et al. (1997) J Biol. Chem. 2:19518. All of which are herein incorporated by reference.

Polynucleotides for use in the methods disclosed herein may be naturally occurring, such as allelic variants, homologs, orthologs, or may be constructed by recombinant DNA methods or by chemical synthesis. Alternatively, the variant polypeptides may be non-naturally occurring and made by techniques known in the art, including mutagenesis. Polynucleotide variants may contain nucleotide substitutions, deletions, inversions and insertions.

As shown in Example 8, SLC encoding nucleic acid molecules can be inserted into vectors and used as gene therapy vectors. In addition to the illustrative adenoviral vectors disclosed herein, a wide range of other host-vector systems suitable for the expression of SLC proteins or fragments thereof are available, see for example, Sambrook et al., 1989, Current Protocols in Molecular Biology, 1995, supra. Gene therapy vectors can be delivered to a subject by, for example, intravenous injection, local administration (U.S. Pat. No. 5,328,470), implantation or by stereotactic injection (see e.g., Chen et al., PNAS 91:3054-3057 (1994)). Vectors for expression in mammalian hosts are disclosed in Wu et al. (1991) J. Biol. Chem. 266:14338; Wu and Wu (1988) J. Biol. Chem. 263:14621; and Zenke et al. (1990) Proc. Nat'l. Acad Sci. USA 87:3655. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Preferred for use in the present invention are adenovirus vectors, and particularly tetracycline-controlled adenovirus vectors. These vectors may be employed to deliver and express a wide variety of genes, including, but not limited to cytokine genes such as those of the interferon gene family and the interleukin gene family.

A preferred method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct in host cells with complementary packaging functions and (b) to ultimately express a heterologous gene of interest that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences (Grunhaus and Horwitz, 1992). In contrast to retrovirus, the adenoviral infection of host cells does not result in chromosomal integration because wild-type adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In a current system, recombinant adenovirus is generated from homologous recombination between a shuttle vector and a master plasmid which contains the backbone of the adenovirus genome. Due to the possible recombination between the backbone of the adenovirus genome, and the cellular DNA of the helper cells which contain the missing portion of the viral genome, wild-type adenovirus may be generated from this process. Therefore, it is critical to isolate a single clone of virus from an individual plaque and examine its genomic structure.

Generation and propagation of most adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome (Jones and Shenk, 1978), the current adenovirus vectors, with the help of 293 cells, carry foreign DNA in either the E1, the E3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of most adenovirus vectors is at least 7.5 kb, or about 15% of the total length of the vector. More than 80% of the adenovirus viral genome remains in the vector backbone.

Gene transfer in vivo using recombinant E1-deficient adenoviruses results in early and late viral gene expression that may elicit a host immune response, thereby limiting the duration of transgene expression and the use of adenoviruses for gene therapy. In order to circumvent these potential problems, the prokaryotic Cre-loxP recombination system has been adapted to generate recombinant adenoviruses with extended deletions in the viral genome in order to minimize expression of immunogenic and/or cytotoxic viral proteins.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Recently, Racher et al., (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for I to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

In some cases, adenovirus mediated gene delivery to multiple cell types has been found to be much less efficient compared to epithelial derived cells. A new adenovirus, AdPK, has been constructed to overcome this inefficiency (Wickham et al., 1996), AdPK contains a heparin-binding domain that targets the virus to heparin-containing cellular receptors, which are broadly expressed in many cell types. Therefore, AdPK delivers genes to multiple cell types at higher efficiencies than unmodified adenovirus, thus improving gene transfer efficiency and expanding the tissues amenable to efficient adenovirus mediated gene therapy.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the foreign gene expression cassette at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect (Brough et al., 1996).

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No severe side effects have been reported in studies of vaccination with wild-type adenovirus (Couch et al., 1963; Top et al., 1971), demonstrating their safety and therapeutic potential as in viva gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1991; Rich et al., 1993). Studies in administering recombinant adenovirus to different tissues include trachea instillation (Rosenfeld et al., 1991; 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing human primary cells.

Adeno-associated virus (AAV) is also an attractive system for use in construction of vectors for delivery of and expression of genes as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994a; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991), rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzvczka, 1992).

Typically, recombinant AAV (rAAV) virus is made by cotransfecting a plasmid containing the gene of interest flanked by the two AAV terminal repeats (McLaughlin et al., 1988: Samulski et al., 1989; each incorporated herein by reference) and an expression plasmid containing the wild type AAV coding sequences without the terminal repeats, for example plM4S (McCarty et al., 1991; incorporated herein by reference). The cells are also infected or transfected with adenovirus or plasmids carrying the adenovirus genes required for AAV helper function, rAAV virus stocks made in such fashion are contaminated with adenovirus which must be inactivated by heat shock or physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

In particular aspects of the present invention, delivery of selected genes to target cells through the use of retrovirus infection will be desired. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988: Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

In some cases, the restricted host-cell range and low titer of retroviral vectors can limit their use for stable gene transfer in eukaryotic cells. To overcome these potential difficulties, a murine leukemia virus-derived vector has been developed in which the retroviral envelope glycoprotein has been completely replaced by the G glycoprotein of vesicular stomatitis virus (Burns et al., 1993). These vectors can be concentrated to extremely high titers (109 colony forming units/ml), and can infect cells that are ordinarily resistant to infection with vectors containing the retroviral envelope protein. These vectors may facilitate gene therapy model studies and other gene transfer studies that require direct delivery of vectors in vivo.

Other viral vectors may be employed for construction of expression vectors in the present invention. Vectors derived from viruses such as vaccinia virus (Ridgeway, 1988; Baichwal and Sugden, 1986; Coupar et al., 1988), sindbis virus and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. (1991) recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

The methods of the present invention may be combined with any other methods generally employed in the treatment of the particular disease or disorder that the patient exhibits. For example, in connection with the treatment of solid tumors, the methods of the present invention may be used in combination with classical approaches, such as surgery, radiotherapy and the like. So long as a particular therapeutic approach is not known to be detrimental in itself, or counteracts the effectiveness of the SLC therapy, its combination with the present invention is contemplated. When one or more agents are used in combination with SLC therapy, there is no requirement for the combined results to be additive of the effects observed when each treatment is conducted separately, although this is evidently desirable, and there is no particular requirement for the combined treatment to exhibit synergistic effects, although this is certainly possible and advantageous.

In terms of surgery, any surgical intervention may be practiced in combination with the present invention. In connection with radiotherapy, any mechanism for inducing DNA damage locally within tumor cells is contemplated, such as y-irradiation, X-rays, UV-irradiation, microwaves and even electronic emissions and the like. The directed delivery of radioisotopes to tumor cells is also contemplated, and this may be used in connection with a targeting antibody or other targeting means. Cytokine therapy also has proven to be an effective partner for combined therapeutic regimens. Various cytokines may be employed in such combined approaches. Examples of cytokines include IL-1α, IL-1β, IL-2, IL-3, IL-4, IL-5, IL-6, IL-7, IL-8, IL-9, IL-10, IL-11, IL-12, IL-13, TGF-β, GM-CSF, M-CSF, TNFα, TNFβ, LAF, TCGF, BCGF, TRF, BAF, BDG, MP, LIF, OSM, TMF, PDGF, IFN-α, IFN-β, IFN-γ. Cytokines are administered according to standard regimens, consistent with clinical indications such as the condition of the patient and relative toxicity of the cytokine. Below is an exemplary, but in no way limiting, table of cytokine genes contemplated for use in certain embodiments of the present invention.

TABLE A

| Cytokine | Reference |
|---|---|
| human IL-1α | March et al., Nature, 315: 641, 1985 |
| murine IL-1α | Lomedico et al., Nature, 312: 458, 1984 |
| human IL-1β | March et al., Nature, 315: 641, 1985; Auron et al., Proc. Natl. Acad. Sci. USA, |
| | 81: 7907, 1984 |
| Murine IL-1β | Gray, J. Immunol., 137L3644m 19861 Tekfirdm Nucl. Acids Res., 14: 9955, 1986 |
| human IL-1ra | Eisenberg et al., Nature, 343: 341, 1990 |
| human IL-2 | Taniguchi et al., Nature, 302: 305, 1983; Maeda et al., Biochem. Biophys, Res. |
| | Commun., 115: 1040, 1983 |
| human IL-2 | Taniguchi et al., Nature, 302: 305, 1983 |
| human IL-3 | Yang et al., Cell, 47: 3, 1986 |
| murine IL-3 | Yokota et al., Proc. Natl. Acad. Sci. USA, 81: 1070, 1984; Fung et al., Nature, |
| | 307: 233, 1984; Miyatake et al., Proc. Natl. Acad. Sci. USA, 82: 316, 1985 |
| human IL-4 | Yokota et al., Proc. Natl. Acad. Sci. USA, 83: 5894, 1986 |
| murine IL-4 | Norma et al., Nature, 319: 640, 1986; Lee et al., Proc. Natl. Acad. Sci. USA, |
| | 83: 2061, 1986 |
| human IL-5 | Azuma et al., Nucl. Acids Res., 14: 9149, 1986 |
| murine IL-5 | Kinashi et al., Nature, 324: 70, 1986; Mizuta et al., Growth Factors, 1: 51, 1988 |
| human IL-6 | Hirona et al., Nature, 324: 73, 1986 |
| murine IL-6 | Van Snick et al., Eur. J. Immunol., 18: 193, 1988 |
| human IL-7 | Goodwin et al., Proc. Natl. Acad. Sci. USA, 86: 302, 1989 |
| murine IL-7 | Namen et al., Nature, 333: 571, 1988 |
| human IL-8 | Schmid et al., J. Immunol., 139: 250, 1987; Matsushima et al, J. Exp. Med., |
| | 167: 1883, 1988; Lindley et al., Proc. Natl Acad. Sci. USA, 85: 9199, 1988 |
| human IL-9 | Renauld et al., J. Immunol., 144: 4235, 1990 |
| murine IL-9 | Renauld et al., J. Immunol., 144: 4235, 1990 |
| human Angiogenin | Kurachi et al., Biochemistry, 24: 5494, 1985 |
| human GROα | Richmond et al., EMBO J, 7: 2025, 1988 |
| murine MIP-1α | Davatelis et al., J. Exp. Med., 167: 1939, 1988 |
| murine MIP-1β | Sherry et al., J. Exp. Med., 167: 2251, 1988 |
| human MIF | Weiser et al., Proc. Natl. Acad. Sci. USA, 86: 7522, 1989 |
| human G-CSF | Nagata et al., Nature, 319: 415, 1986; Souza et al., Science, 232: 61, 1986 |
| human GM-CSF | Cantrell et al., Proc. Natl. Acad. Sci. USA, 82: 6250, 1985; Lee et al., Proc. Natl. |
| | Acad. Sci. USA, 82: 4360, 1985; Wong et al., Science, 228: 810, 1985 |
| murine GM-CSF | Gough et al., EMBO J., 4: 645, 1985 |
| human M-CSF | Wong, Science, 235: 1504, 1987; Kawasaki, Science, 230: 291, 1985; Ladner, EMBO |
| | J., 6: 2693, 1987 |
| human EGF | Smith et al., Nucl. Acids Res., 10: 4467, 1982; Bell et al., Nucl. Acids Res., 14: 8427, |
| | 1986 |
| human TGF-α | Derynck et al., Cell, 38: 287, 1984 |
| human FGF acidic | Jaye et al., Science, 233: 541, 1986; Gimenez-Gallego et al., Biochem. Biophys. Res. |
| | Commun., 138: 611, 1986; Harper et al., Biochem., 25: 4097, 1986 |
| human β-ECGF | Jaye et al., Science, 233: 541, 1986 |
| human FGF basic | Abraham et al., EMBO J., 5: 2523, 1986; Sommer et al., Biochem. Biophys. Res. |
| | Comm., 144: 543, 1987 |
| murine IFN-β | Higashi et al., J. Biol. Chem., 258: 9522, 1983; Kuga, Nucl. Acids Res., 17: 3291, |
| | 1989 |
| human IFN-γ | Gray et al., Nature, 295: 503, 1982; Devos et al., Nucl. Acids Res., 10: 2487, 1982; |
| | Rinderknecht, J. Biol. Chem. 259: 6790, 1984 |
| human IGF-I | Jansen et al., Nature, 306: 609, 1983; Rotwein et al., J. Biol. Chem., 261: 4828, 1986 |
| human IGF-II | Bell et al., Nature, 310: 775, 1984 |
| human β-NGF chain | Ullrich et al., Nature, 303: 821, 1983 |
| human PDGF A chain | Betsholtz et al., Nature, 320: 695, 1986 |
| human PDGF B chain | Johnsson et al., EMBO J, 3: 921, 1984; Collins et al., Nature, 316: 748, 1985 |
| human TGF-β1 | Derynck et al., Nature, 316: 701, 1985 |
| human TNF-α | Pennica et al., Nature, 312: 724, 1984; Fransen et al., Nucl. Acids Res., 13: 4417, |
| | 1985 |
| human TNF-β | Gray et al., Nature, 312: 721, 1984 |
| murine TNF-β | Gray et al., Nucl. Acids Res., 15: 3937, 1987 |

Compositions of the present invention can have an effective amount of an engineered virus or cell for therapeutic administration in combination with an effective amount of a compound (second agent) that is a chemotherapeutic agent as exemplified below. Such compositions will generally be dissolved or dispersed in a pharmaceutically acceptable carrier or aqueous medium. A wide variety of chemotherapeutic agents may be used in combination with the therapeutic genes of the present invention. These can be, for example, agents that directly cross-link DNA, agents that intercalate into DNA, and agents that lead to chromosomal and mitotic aberrations by affecting nucleic acid synthesis.

A variety of chemotherapeutic agents are intended to be of use in the combined treatment methods disclosed herein. Chemotherapeutic agents contemplated as exemplary include, e.g., etoposide (VP-16), adriamycin, 5-fluorouracil (5FU), camptothecin, actinomycin-D, mitomycin C, cisplatin (CDDP) and even hydrogen peroxide.

As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, agents such as cisplatin, and other DNA alkylating may be used. Cisplatin has been widely used to treat cancer, with efficacious doses used in clinical applications of 20 mg/in² for 5 days every three weeks for a total of three courses. Cisplatin is not absorbed orally and must therefore be delivered via injection intravenously, subcutaneously, intratumorally or intraperitoneally.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Further useful agents include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/in2 at 21 day intervals for adriamycin, to 35-50 mg/in2 for etoposide intravenously or double the intravenous dose orally.

Agents that disrupt the synthesis and fidelity of polynucleotide precursors may also be used. Particularly useful are agents that have undergone extensive testing and are readily available. As such, agents such as 5-fluorouracil (5-FU) are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Plant alkaloids such as taxol are also contemplated for use in certain aspects of the present invention. Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

Exemplary chemotherapeutic agents that are useful in connection with combined therapy are listed in Table B. Each of the agents listed therein are exemplary and by no means limiting. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

TABLE B
Table 4
Chemotherapeutic Agents Useful In Neoplastic Disease

| | | Nonproprietary Names | |
|---|---|---|---|
| Class | Type Of Agent | (Other Names) | Disease |
| Alkylating | Nitrogen | Mechlorethamine | Hodgkin's |
| Agents | Mustards | (HN₂) | disease, non- |
| | | | Hodgkin's |
| | | | lymphomas |
| | | Cyclophosphamide | Acute and |
| | | Ifosfamide | chronic |
| | | | lymphocytic |
| | | | leukemias, |
| | | | Hodgkin's |
| | | | disease, non- |
| | | | Hodgkin's |
| | | | lymphomas, |
| | | | multiple |
| | | | myeloma, |
| | | | neuroblastoma, |
| | | | breast, ovary, |
| | | | lung, Wilms' |
| | | | tumor, cervix, |
| | | | testis, soft- |
| | | | tissue |
| | | | sarcomas |
| | | Melphalan (L- | Multiple |
| | | sarcolysin) | myeloma, |
| | | | breast, ovary |
| | | Chlorambucil | Chronic |
| | | | lymphocytic |
| | | | leukemia, |
| | | | primary |
| | | | macroglobuline |
| | | | mia, Hodgkin's |
| | | | disease, non- |
| | | | Hodgkin's |
| | | | lymphomas |
| | Ethylenimenes | Hexamethylmelamine | Ovary |
| | and | Thiotepa | Bladder, |
| | Methylmelamines | | breast, ovary |
| | Alkyl Sulfonates | Busulfan | Chronic |
| | | | granulocytic |
| | | | leukemia |
| | Nitrosoureas | Carmustine (BCNU) | Hodgkin's |
| | | | disease, non- |
| | | | Hodgkin's |
| | | | lymphomas, |
| | | | primary brain |
| | | | tumors, |
| | | | multiple |
| | | | myeloma, |
| | | | malignant |
| | | | melanoma |
| | | Lomustine (CCNU) | Hodgkin's |
| | | | disease, non- |
| | | | Hodgkin's |
| | | | lymphomas, |
| | | | primary brain |
| | | | tumors, small- |
| | | | cell lung |
| | | Semustine (methyl- | Primary brain |
| | | CCNU) | tumors, |
| | | | stomach, colon |
| | | Streptozocin | Malignant |
| | | (Streptozotocin) | pancreatic |
| | | | insulinoma, |
| | | | malignant |
| | | | carcinoid |
| | Triazines | Dacarbazine (DTIC; | Malignant |
| | | dimethyltrizenoimidazole- | melanoma, |
| | | carboxamide) | Hodgkin's |
| | | | disease, soft- |
| | | | tissue |
| | | | sarcomas |
| Antimetabolites | Folic Acid | Methotrexate | Acute |
| | Analogs | (amethopterin) | lymphocytic |
| | | | leukemia, |
| | | | choriocarcinoma, |
| | | | mycosis |
| | | | fungoides, |
| | | | breast, head |
| | | | and neck, |
| | | | lung, |
| | | | osteogenic |
| | | | sarcoma |
| | Pyrimidine | Fluouracil (5- | Breast, colon, |
| | Analogs | fluorouracil; | stomach, |
| | | 5-FU) | pancreas, |
| | | Floxuridine | ovary, head |
| | | (fluoro- | and neck, |
| | | deoxyuridine; | urinary |
| | | FUdR) | bladder, |
| | | | premalignant |
| | | | skin lesions |
| | | | (topical) |
| | | Cytarabine | Acute |
| | | (cytosine | granulocytic |
| | | arabinoside) | and acute |
| | | | lymphocytic |
| | | | leukemias |
| | Purine Analogs | Mercaptopurine | Acute |
| | and Related | (6- | lymphocytic, |
| | Inhibitors | mercaptopurine; | acute |
| | | 6-MP) | granulocytic |
| | | | and chronic |
| | | | granulocytic |
| | | | leukemias |
| | | Thioguanine | Acute |
| | | (6-thioguanine; | granulocytic, |
| | | TG) | acute |
| | | | lymphocytic |
| | | | and chronic |
| | | | granulocytic |
| | | | leukemias |
| | | Pentostatin | Hairy cell |
| | | (2- | leukemia, |
| | | deoxycoformycin) | mycosis |
| | | | fungoides, |
| | | | chronic |
| | | | lymphocytic |
| | | | leukemia |
| Natural | Vinca Alkaloids | Vinblastine (VLB) | Hodgkin's |
| Products | | | disease, non- |
| | | | Hodgkin's |
| | | | lymphomas, |
| | | | breast, testis |
| | | Vincristine | Acute |
| | | | lymphocytic |
| | | | leukemia, |
| | | | neuroblastoma, |
| | | | Wilms' tumor, |
| | | | rhabdomyosarcoma, |
| | | | Hodgkin's |
| | | | disease, non- |
| | | | Hodgkin's |
| | | | lymphomas, |
| | | | small-cell |
| | | | lung |
| | Epipodophyllotoxins | Etoposide (VP16) | Testis, small- |
| | | Tertiposide | cell lung and |
| | | | other lung, |
| | | | breast, |
| | | | Hodgkin's |
| | | | disease, non- |
| | | | Hodgkin's |
| | | | lymphomas, |
| | | | acute |
| | | | granulocytic |
| | | | leukemia, |
| | | | Kaposi's |
| | | | sarcoma |
| | Antibiotics | Dactinomycin | Choriocarcinoma, |
| | | (actinomycin D) | Wilms' |
| | | | tumor, |
| | | | rhabdomyosarcoma, |
| | | | testis, |
| | | | Kaposi's |
| | | | sarcoma |
| | | Daunorubicin | Acute |
| | | (daunomycin; | granulocytic |
| | | rubidomycin) | and acute |
| | | | lymphocytic |
| | | | leukemias |
| | | Doxorubicin | Soft-tissue, |
| | | | osteogenic and |
| | | | other |
| | | | sarcomas; |
| | | | Hodgkin's |
| | | | disease, non- |
| | | | Hodgkin's |
| | | | lymphomas, |
| | | | acute |
| | | | leukemias, |
| | | | breast, |
| | | | genitourinary, |
| | | | thyroid, lung, |
| | | | stomach, |
| | | | neuroblastoma |
| | | Bleomycin | Testis, head |
| | | | and neck, |
| | | | skin, |
| | | | esophagus, |
| | | | lung and |
| | | | genitourinary |
| | | | tract; |
| | | | Hodgkin's |
| | | | disease, non- |
| | | | Hodgkin's |
| | | | lymphomas |
| | | Plicamycin | Testis, |
| | | (mithramycin) | malignant |
| | | | hypercalcemia |
| | | Mitomycin | Stomach, |
| | | (mitomycin C) | cervix, colon, |
| | | | breast, |
| | | | pancreas, |
| | | | bladder, head |
| | | | and neck |
| | Enzymes | L-Asparaginase | Acute |
| | | | lymphocytic |
| | | | leukemia |
| | Biological | Interferon alfa | Hairy cell |
| | Response | | leukemia, |
| | Modifiers | | Kaposi's |
| | | | sarcoma, |
| | | | melanoma, |
| | | | carcinoid, |
| | | | renal cell, |
| | | | ovary, |
| | | | bladder, non- |
| | | | Hodgkin's |
| | | | lymphomas, |
| | | | mycosis |
| | | | fungoides, |
| | | | multiple |
| | | | myeloma, |
| | | | chronic |
| | | | granulocytic |
| | | | leukemia |
| Miscellaneous | Platinum | Cisplatin (cis-DDP) | Testis, ovary, |
| Agents | Coordination | Carboplatin | bladder, head |
| | Complexes | | and neck, |
| | | | lung, thyroid, |
| | | | cervix, |
| | | | endometrium, |
| | | | neuroblastoma, |
| | | | osteogenic |
| | | | sarcoma |
| | Anthracenedione | Mitoxantrone | Acute |
| | | | granulocytic |
| | | | leukemia, |
| | | | breast |
| | Substituted Urea | Hydroxyurea | Chronic |
| | | | granulocytic |
| | | | leukemia, |
| | | | polycythemia |
| | | | vera, essental |
| | | | thrombocytosis, |
| | | | malignant |
| | | | melanoma |
| | Methyl Hydrazine | Procarbazine | Hodgkin's |
| | Derivative | (N- | disease |
| | | methylhydrazine, | |
| | | MIH) | |
| | Adrenocortical | Mitotane | Adrenal cortex |
| | Suppressant | (o.p′-DDD) | |
| | | Aminoglutethimide | Breast |
| Hormones | Adrenocorticosteroids | Prednisone (several | Acute and |
| and | | other equivalent | chronic |
| Antagonists | | preparations | lymphocytic |
| | | available) | leukemias, |
| | | | non-Hodgkin's |
| | | | lymphomas, |
| | | | Hodgkin's |
| | | | disease, |
| | | | breast |
| | Progestins | Hydroxyprogesterone | Endometrium, |
| | | caproate | breast |
| | | Medroxyprogesterone | |
| | | acetate | |
| | | Megestrol acetate | |
| | Estrogens | Diethylstilbestrol | Breast, |
| | | Ethinyl estradiol | prostate |
| | | (other preparations | |
| | | available) | |
| | Antiestrogen | Tamoxifen | Breast |
| | Androgens | Testosterone | Breast |
| | | propionate | |
| | | Fluoxymesterone | |
| | | (other preparations | |
| | | available) | |
| | Antiandrogen | Flutamide | Prostate |
| | Gonadotropin- | Leuprolide | Prostate |
| | releasing | | |
| | hormone analog | | |

The SLC polypeptides, SLC polypeptide variants, SLC polypeptide fragments, SLC polynucleotides encoding said polypeptides, variants and fragments, and the SLC agents useful in the methods of the invention can be incorporated into pharmaceutical compositions suitable for administration into a mammal. The term “mammal” as used herein refers to any mammal classified as a mammal, including humans, cows, horses, dogs and cats. In a preferred embodiment of the invention, the mammal is a human. Such compositions typically comprise at least one SLC polypeptide, SLC polypeptide variant, SLC polypeptide fragment, SLC polynucleotide encoding said polypeptide, variant or fragment, an SLC agent, or a combination thereof, and a pharmaceutically acceptable carrier. Methods for formulating the SLC compounds of the invention for pharmaceutical administration are known to those of skill in the art. See, for example, Remington: The Science and Practice of Pharmacy, 19^{th} Edition, Gennaro (ed.) 1995, Mack Publishing Company, Easton, Pa.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the, invention is formulated to be compatible with its intended route of administration.

The route of administration will vary depending on the desired outcome. Generally for initiation of an immune response, injection of the agent at or near the desired site of inflammation or response is utilized. Alternatively other routes of administration may be warranted depending upon the disease condition. That is, for suppression of neoplastic or tumor growth, injection of the pharmaceutical composition at or near the tumor site is preferred. Alternatively, for prevention of graft rejection, systemic administration maybe used. Likewise, for the treatment or prevention of autoimmune diseases systemic administration may be preferred. Examples of routes of systemic administration include parenteral, e.g., intravenous, intradermal, subcutaneous, oral (e.g., inhalation) transdermal (topical), transmucosal, and rectal administration. Solutions or suspensions used for parenteral, intradermal, or subcutaneous application can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose.

In one embodiment, the pharmaceutical composition can be delivered via slow release formulation or matrix comprising SLC protein or DNA constructs suitable for expression of SLC protein into or around a site within the body. In this manner, a transient lymph node can be created at a desired implant location to attract dendritic cells and T cells initiating an immune response.

The pharmaceutical compositions can be included in a container, pack, or dispenser together with instructions for administration. That result can be reduction and/or alleviation of the signs, symptoms, or causes of a disease or any other desired alteration of a biological system. The pharmaceutical compositions of the invention, comprising SLC polypeptides, SLC polypeptide variants, SLC polypeptide fragments, polynucleotides encoding said SLC polypeptides, variants and fragments, as well as SLC agents, as defined above, are administered in therapeutically effective amounts. The “therapeutically effective amount” refers to a nontoxic dosage level sufficient to induce a desired biological result (e.g. the enhancement of an immune response). In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing tumor burden or volume, the time to disease progression (TTP) and/or determining the response rates (RR).

Amounts for administration may vary based upon the desired activity, the diseased state of the mammal being treated, the dosage form, method of administration, patient factors such as age, sex, and severity of disease. It is recognized that a therapeutically effective amount is provided in a broad range of concentrations. Such range can be determined based on binding assays, chemotaxis assays, and in vivo assays.

Regimens of administration may vary. A single injection or multiple injections of the agent may be used. Likewise, expression vectors can be used at a target site for continuous expression of the agent. Such regimens will vary depending on the severity of the disease and the desired outcome. In a preferred embodiment, an SLC or SLC composition is injected directly into the tumor or into a peritumor site. By peritumor site is meant a site less than about 15 cm from an outer edge of the tumor. In a highly preferred embodiment, an SLC or SLC composition is injected into an lymph node that is proximal to the tumor. SLC administration may be to one or more sites. Preferably, SLC administration is at multiple sites within a tumor and/or surrounding a tumor.

The SLC polypeptide is preferably administered to the mammal in a carrier; preferably a pharmaceutically-acceptable carrier. Suitable carriers and their formulations are described in Remington's Pharmaceutical Sciences, 16th ed., 1980, Mack Publishing Co., edited by Oslo et al. Typically, an appropriate amount of a pharmaceutically-acceptable salt is used in the formulation to render the formulation isotonic. Examples of the carrier include saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing, for example, the SLC polypeptide, which matrices are in the form of shaped articles, e.g., films, liposomes or microparticles. It will be apparent to those persons skilled in the art that certain carriers may be more preferable depending upon, for instance, the route of administration and concentration of SLC polypeptide being administered.

The SLC polypeptide can be administered to the mammal by injection (e.g., intravenous, intraperitoneal, subcutaneous, intramuscular, intraportal), or by other methods such as infusion that ensure its delivery to the bloodstream in an effective form. The SLC polypeptide may also be administered by isolated perfusion techniques, such as isolated tissue perfusion, to exert local therapeutic effects. Local or intravenous injection is preferred.

Effective dosages and schedules for administering the SLC polypeptides may be determined empirically (e.g. using the models disclosed herein), and making such determinations is within the skill in the art. Those skilled in the art will understand that the dosage of SLC polypeptide that must be administered will vary depending on, for example, the mammal which will receive the SLC polypeptide, the route of administration, the particular type of molecule used (e.g. polypeptide, polynucleotide etc.) used and other drugs being administered to the mammal.

As noted above, the SLC polypeptide may be administered sequentially or concurrently with one or more other therapeutic agents. The amounts of this molecule and therapeutic agent depend, for example, on what type of drugs are used, the pathological condition being treated, and the scheduling and routes of administration but would generally be less than if each were used individually. It is contemplated that the antagonist or blocking SLC antibodies may also be used in therapy. For example, a SLC antibody could be administered to a mammal (such as described above) to block SLC receptor binding.

Following administration of a SLC polypeptide to the mammal, the mammal's physiological condition can be monitored in various ways well known to the skilled practitioner. The therapeutic effects of the SLC polypeptides of the invention can be examined in in vitro assays and using in vivo animal models. A variety of well known animal models can be used to further understand the role of the SLC in the development and pathogenesis of for instance, immune related disease or cancer, and to test the efficacy of the candidate therapeutic agents. The in vivo nature of such models makes them particularly predictive of responses in human patients. Animal models of immune related diseases include both non-recombinant and recombinant (transgenic) animals. Non-recombinant animal models include, for example, rodent, e.g., murine models. Such models can be generated by introducing cells into syngeneic mice using standard techniques, e.g. subcutaneous injection, tail vein injection, spleen implantation, intraperitoneal implantation, and implantation under the renal capsule.

In a further embodiment of the invention, there are provided articles of manufacture and kits containing materials useful for treating pathological conditions or detecting or purifying SLC. The article of manufacture comprises a container with a label. Suitable containers include, for example, bottles, vials, and test tubes. The containers may be formed from a variety of materials such as glass or plastic. The container holds a composition having an active agent which is effective for treating pathological conditions such as cancer. The active agent in the composition is preferably SLC. The label on the container indicates that the composition is used for treating pathological conditions or detecting or purifying SLC, and may also indicate directions for either in vivo or in vitro use, such as those described above.

The kit of the invention comprises the container described above and a second container comprising a buffer. It may further include other materials desirable from a commercial and user standpoint, including other buffers, diluents, filters, needles, syringes, and package inserts with instructions for use.

### C. Illustrative Embodiments of the Invention

The invention disclosed herein has a number of embodiments. A preferred embodiment of the invention is a method of effecting or modulating cytokine expression (e.g. changing an existing cytokine profile) in a mammal or in a population of cells derived from a mammal by exposing the population of cells to an amount of secondary lymphoid tissue chemokine (SLC) polypeptide sufficient to inhibit the growth of syngeneic tumor cells such as the spontaneous carcinoma cells that arise in the transgenic mouse model described herein. As disclosed herein, because the syngeneic models disclosed herein demonstrate how the addition of SLC coordinately modulates cytokine expression and inhibits the growth of the tumor cells, observations of these phenomena (modulation of cytokine expression and inhibition of tumor growth) can be used in cell based assays designed to assess the effects of potential immunostimulatory or immunoinhibitory test compounds. For example the disclosure provided herein allows one to examine the effects that test compound has on the ability of SLC to modulate cytokine expression and to identify compounds which modulate cytokine profiles in an advantageous manner.

The methods described herein can be employed in a number of contexts. For example the method described above can be practiced serially as the effects of compounds that have the ability modulate the cytokine profiles is examined. In one such embodiment of the invention, the cytokine profile (and/or inhibition of tumor growth) in response to SLC in a given cancer model is first examined to determine the effects of SLC in that specific context. The results of such assays can then be compared to the effects that SLC has on a known cancer model such as the transgenic mouse model described herein in order to confirm the effects of SLC in that model. A variation of the method can then be repeated using a test compound in place of SLC and the cytokine profile with the response to the test compound in the model then being examined to identify molecules which can produce physiological effects that are similar or dissimilar to SLC (e.g. modulate cytokine profile and/or inhibition of tumor growth in a specific way). In a related embodiment SLC and a test compound can be added simultaneously to see if the test compound can modulate the effects of SLC in a manner that may have some clinical applicability, for example to modulate the cytokine profile in a manner that enhances the inhibition of tumor growth, allows inhibition of growth with fewer side effects etc. As these models measure and compare both cytokine profiles and/or inhibition of tumor growth and because these are shown herein to be linked, the models provide internal references which facilitates the identification new molecules of interest and the dissection their effects on cellular physiology.

These methods provide a particularly useful clinical model because they parallel methods of treatment. Specifically, treating a cancer with SLC entails a method of effecting or modulating cytokine expression (e.g. changing the existing cytokine profile) in a mammal or in a certain population of cells derived from a mammal by exposing the population of cells to an amount of secondary lymphoid tissue chemokine (SLC) polypeptide sufficient to inhibit the growth of syngeneic tumor cells. In such clinical contexts, the effects of SLC in a given system can be observed or monitored in a number of ways, for example, the effects of SLC can be observed by the evaluation of a change in a cytokine profile, an evaluation the inhibition of tumor growth or tumor killing (e.g. by observing a reduction in tumor size and/or a reduction in the severity of symptoms associated with the tumor and/or tumor growth), an increased survival rate (as observed with the transgenic mouse model disclosed herein) and the like.

A specific embodiment of this embodiment of the invention is a method of effecting an increase in the expression of Interferon-γ (IFN-γ) polypeptide and a decrease in the expression of Transforming Growth Factor-β (TGF-β) polypeptide in a population of syngeneic mammalian cells including CD8 positive T cells, CD4 positive T cells, Antigen Presenting Cells and tumor cells comprising exposing the population of cells to an amount of secondary lymphoid tissue chemokine (SLC) polypeptide sufficient to inhibit the growth of the tumor cells and then repeating this method and additionally exposing the population of cells to a test compound consisting of a small molecule or polypeptide agent. The data from these assays can then be compared to observe effect that the test compound has on the expression of IFN-γ polypeptide or the expression of TGF-β polypeptide.

Any molecule known in the art can be tested for its ability to mimic or modulate (increase or decrease) the activity of SLC as detected by a change in the level of certain cytokines. For identifying a molecule that mimics or modulates SLC activity, candidate molecules can be directly provided to a cell or test subject in vivo or in vitro in order to detect the change in cytokine expression. Moreover, any lead activator or inhibitor structure known in the art can be used in conjunction with the screening and treatment methods of the invention. Such structures may be used, for example, to assist in the development of activators and/or inhibitors of SLC.

This embodiment of the invention is well suited to screen chemical libraries for molecules which modulate, e.g., inhibit, antagonize, or agonize or mimic, the activity of SLC as measured by the change in cytokine levels. The chemical libraries can be peptide libraries, peptidomimetic libraries, chemically synthesized libraries, recombinant, e.g., phage display libraries, and in vitro translation-based libraries, other non-peptide synthetic organic libraries, etc.

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

The libraries can be constrained or semirigid (having some degree of structural rigidity), or linear or nonconstrained. The library can be a cDNA or genomic expression library, random peptide expression library or a chemically synthesized random peptide library, or non-peptide library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al., 1991, Nature 354:84-86, which describes mixtures of free hexapeptides in which the first and second residues in each peptide were individually and specifically defined; Lam et al., 1991, Nature 354:82-84, which describes a “one bead, one peptide” approach in which a solid phase split synthesis scheme produced a library of peptides in which each bead in the collection had immobilized thereon a single, random sequence of amino acid residues; Medynski, 1994, Bio/Technology 12:709-710, which describes split synthesis and T-bag synthesis methods; and Gallop et al., 1994, J. Medicinal Chemistry 37(9):1233-1251. Simply by way of other examples, a combinatorial library may be prepared for use, according to the methods of Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; or Salmon et al., 1993, Proc. Natl. Acad. Sci. USA 90:11708-11712. PCT Publication No. WO 93/20242 and Brenner and Lerner, 1992, Proc. Natl. Acad. Sci. USA 89:5381-5383 describe “encoded combinatorial chemical libraries,” that contain oligonucleotide identifiers for each chemical polymer library member.

In a preferred embodiment, the library screened is a biological expression library that is a random peptide phage display library, where the random peptides are constrained (e.g., by virtue of having disulfide bonding).

Further, more general, structurally constrained, organic diversity (e.g., nonpeptide) libraries, can also be used. By way of example, a benzodiazepine library (see e.g., Bunin et al., 1994, Proc. Natl. Acad. Sci. USA 91:4708-4712) may be used.

Conformationally constrained libraries that can be used include but are not limited to those containing invariant cysteine residues which, in an oxidizing environment, cross-link by disulfide bonds to form cysteines, modified peptides (e.g., incorporating fluorine, metals, isotopic labels, are phosphorylated, etc.), peptides containing one or more non-naturally occurring amino acids, non-peptide structures, and peptides containing a significant fraction of (-carboxyglutamic acid.

Libraries of non-peptides, e.g., peptide derivatives (for example, that contain one or more non-naturally occurring amino acids) can also be used. One example of these are peptoid libraries (Simon et al., 1992, Proc. Natl. Acad. Sci. USA 89:9367-9371). Peptoids are polymers of non-natural amino acids that have naturally occurring side chains attached not to the alpha carbon but to the backbone amino nitrogen. Since peptoids are not easily degraded by human digestive enzymes, they are advantageously more easily adaptable to drug use. Another example of a library that can be used, in which the amide functionalities in peptides have been permethylated to generate a chemically transformed combinatorial library, is described by Ostresh et al., 1994, Proc. Natl. Acad. Sci. USA 91:11138-11142).

The members of the peptide libraries that can be screened according to the invention are not limited to containing the 20 naturally occurring amino acids. In particular, chemically synthesized libraries and polysome based libraries allow the use of amino acids in addition to the 20 naturally occurring amino acids (by their inclusion in the precursor pool of amino acids used in library production). In specific embodiments, the library members contain one or more non-natural or non-classical amino acids or cyclic peptides. Non-classical amino acids include but are not limited to the D-isomers of the common amino acids, ″-amino isobutyric acid, 4-aminobutyric acid, Abu, 2-amino butyric acid; (-Abu, -Ahx, 6-amino hexanoic acid; Aib, 2-amino isobutyric acid; 3-amino propionic acid; ornithine; norleucine; norvaline, hydroxyproline, sarcosine, citrulline, cysteic acid, t-butylglycine, t-butylalanine, phenylglycine, cyclohexylalanine, β-alanine, designer amino acids such as β-methyl amino acids, C″-methyl amino acids, N″-methyl amino acids, fluoro-amino acids and amino acid analogs in general. Furthermore, the amino acid can be D (dextrorotary) or L (levorotary).

In a specific embodiment, fragments and/or analogs of proteins of the invention, especially peptidomimetics, are screened for activity as competitive or non-competitive inhibitors of activity.

In another embodiment of the present invention, combinatorial chemistry can be used to identify modulators. Combinatorial chemistry is capable of creating libraries containing hundreds of thousands of compounds, many of which may be structurally similar. While high throughput screening programs are capable of screening these vast libraries for affinity for known targets, new approaches have been developed that achieve libraries of smaller dimension but which provide maximum chemical diversity. (See e.g., Matter, 1997, Journal of Medicinal Chemistry 40:1219-1229).

One method of combinatorial chemistry, affinity fingerprinting, has previously been used to test a discrete library of small molecules for binding affinities for a defined panel of proteins. The fingerprints obtained by the screen are used to predict the affinity of the individual library members for other proteins or receptors of interest The fingerprints are compared with fingerprints obtained from other compounds known to react with the protein of interest to predict whether the library compound might similarly react. For example, rather than testing every ligand in a large library for interaction with a complex or protein component, only those ligands having a fingerprint similar to other compounds known to have that activity could be tested. (See, e.g., Kauvar et al., 1995, Chemistry and Biology 2:107-118; Kauvar, 1995, Affinity fingerprinting, Pharmaceutical Manufacturing International. 8:25-28; and Kauvar, Toxic-Chemical Detection by Pattern Recognition in New Frontiers in Agrochemical Immunoassay, D. Kurtz. L. Stanker and J. H. Skerritt. Editors, 1995, AOAC: Washington, D.C., 305-312).

Kay et al., 1993, Gene 128:59-65 (Kay) discloses a method of constructing peptide libraries that encode peptides of totally random sequence that are longer than those of any prior conventional libraries. The libraries disclosed in Kay encode totally synthetic random peptides of greater than about 20 amino acids in length. Such libraries can be advantageously screened to identify complex modulators. (See also U.S. Pat. No. 5,498,538 dated Mar. 12, 1996; and PCT Publication No. WO 94/18318 dated Aug. 18, 1994).

A comprehensive review of various types of peptide libraries can be found in Gallop et al., 1994, J. Med. Chem. 37:1233-1251.

The population of syngeneic mammalian cells used in these methods typically includes CD8 positive T cells (i.e. those T cells expressing the CD8 antigen), CD4 positive T cells (i.e. those T cells expressing the CD8 antigen), Antigen Presenting Cells (APCs) and tumor cells. The term antigen presenting cell refers to cells that constitutively express class II MHC molecules and present stimulatory antigens to T_{H} cells. There are three major classes of cells that function as APCs. These classes are macrophages, dendritic cells and B lymphocytes. Dendritic cells are the most potent among antigen presenting cells and are believed to be indispensable to the initiation of primary immune responses (see, e.g., Lanzavecchia (1993) Science 260: 937 and Grabbe et al., (1995) Immunology Today 16:117). Tumor cells are typically identified through a wide variety of techniques, including but not limited to, palpation, blood analysis, x-ray, NMR and the like. Moreover, a wide variety of diagnostic factors that are known in the art to be associated with cancer may be utilized to identify a tumor cells such as the expression of genes associated with malignancy (e.g. PSA, PSCA, PSM and human glandular kallikrein expression) as well as gross cytological observations (see e.g. Bocking et al., Anal Quant Cytol. 6(2):74-88 (1984); Eptsein, Hum Pathol. 1995 February; 26(2):223-9 (1995); Thorson et al., Mod Pathol. 1998 June; 11(6):543-51; Baisden et al., Am J Surg Pathol. 23(8):918-24 (1999)).

Using the models and methods disclosed herein, one can readily assess how the administration of SLC modulates cytokine profiles in an immune reaction and/or inhibits the growth of various spontaneous tumors. In preferred embodiments of the invention, SLC is administered to modulate cytokine profiles and/or inhibit the growth of spontaneous tumor cells of the adenocarcinoma lineage as is demonstrated herein. As is known in the art, the major forms of lung cancer including adenocarcinoma, squamous cell carcinoma, small cell carcinoma and large cell carcinoma represent a continuum of differentiation within a common cell lineage and express a number of tumor associated antigens (see, e.g. Berger et al., J Clin Endocrinol Metab 1981 53(2): 422-429 and Niho et al., Gan To Kagaku Ryoho 2001: 28(13): 2089-93; Ohshio et al., Tumori 1995 81(1):67-73 and Hamasaki et al., Anticancer Res 2001 21(2A):979-984). Consequently, the shared lineage relationships and antigenic profile provide evidence that SLC will have a closely analogous effect on the growth of these cancers of the lung (i.e. adenocarcinoma related lung cancers).

Preferably this method of effecting or modulating cytokine expression entails increasing the expression of Interferon-γ (IFN-γ, see, e.g. accession nos. AAB59534 and P01580) polypeptides and/or decreasing in the expression of Transforming Growth Factor-β (TGF-β, see, e.g., accession nos. AAA50405 and AAK56116) polypeptides in a population of syngeneic mammalian cells. In preferred methods, the increase in the expression of Interferon-γ (IFN-γ) polypeptides is at least about two-fold and a decrease in the expression of Transforming Growth Factor-β (TGF-β) polypeptides is at least about two-fold as measured by an enzyme linked immunoadsorbent (ELISA) assay. The effects of SLC in a given system can be observed in a number of other ways in addition to the ELISA assays discussed herein. For example, the effects of SLC can be observed by evaluation the inhibition of tumor growth or tumor killing (e.g. by observing a reduction in tumor size), and an increased survival rate (as observed with the transgenic mouse model disclosed herein) etc.

As disclosed herein the addition of SLC to this population of cells effects an increase in Granulocyte-Macrophage colony stimulating factor (GM-CSF, See, e.g. accession nos. gi:2144692 and gi:69708) polypeptides, monokine induced by IFN-γ (MIG, see, e.g. accession nos. P18340 and Q07325) polypeptides, Interleukin-12 (IL-12, see, e.g. accession nos. NP_{—}032377 AAD56385 and AAD56386) polypeptides or IFN-γ inducible protein 10 (see, e.g. accession nos. P02778 and AAA02968) polypeptides; as well as a decrease in Prostaglandin E(2) polypeptides or vascular endothelial growth factor (VEGF, see, e.g. accession nos. NP_{—}003367 and NP_{—}033531) polypeptides. Consequently, preferred methods include those that generate a change in the cytokine profiles of these molecules via the administration of SLC. This modulation of polypeptide expression can be determined by any one of the wide variety of methods that are used in the art for evaluating gene expression such as the ELISA assays disclosed herein. In preferred methods, the increase and/or decrease in the expression of the polypeptides is at least about two-fold as measured by an enzyme linked immunoadsorbent (ELISA) assay. Additional profiling techniques are known in the art (see, e.g., Peale et al., J. Pathol 2001; 195(1):7-19).

The inhibition of tumor growth can be measured by any one of a wide variety of methods known in the art. Preferably wherein the inhibition of the growth of the syngeneic tumor cells is measured by quantification of tumor surface area. In preferred methods the syngeneic tumor cells are spontaneous cancer cells. As disclosed herein, transgenic which express SV40 large TAg transgene under the control of the murine Clara cell-specific promoter develop diffuse bilateral bronchoalveolar carcinoma. This model is but one of many syngeneic animal models of cancer known in the art that can be utilized according to the methods described herein (see, also Hakem et al., Annu. Rev. Genet. 2001; 35:209-41; Mundy Semin. Oncol. 2001 28(4 Suppl 11): 2-8; Sills et al., Toxicol Lett 2001 120(1-3): 1887-198; Kitchin, Toxicol Appl Pharmacol 2001; 172(3):249-61; and D'Angelo et al., J. Neurooncol 2000; 50(1-2):89-98).

In the methods disclosed hereinabove, the syngeneic cells can be exposed to the SLC by a variety of methods, for example by administering SLC polypeptide to a mammal via intratumoral injection, or alternatively administering SLC polypeptide to a mammal via intra-lymph node injection. In yet another mode of administration, an expression vector having a polynucleotide encoding a SLC polypeptide is administered to the mammal and the SLC polypeptide is produced by a syngeneic mammalian cell that has been transduced with an expression vector encoding the SLC polypeptide.

Yet another embodiment of the invention is a method of inhibiting the growth of spontaneous mammalian cancer cells in a population of syngeneic CD8 positive T cells, CD4 positive T cells and Antigen Presenting Cells by exposing the population of cells to an amount of secondary lymphoid tissue chemokine (SLC) polypeptide sufficient to inhibit the growth of the cancer cells. A closely related embodiment of the invention is a method of treating a syngeneic cancer in a mammalian subject comprising administering a therapeutically effective amount of an SLC to the subject. In preferred methods the SLC is human SLC. In highly preferred methods the SLC has the polypeptide sequence shown in SEQ ID NO: 1. Preferably, the SLC polypeptide is administered to a mammal via intratumoral injection, or via intra-lymph node injection. In yet another mode of administration, an expression vector having a polynucleotide encoding a SLC polypeptide is administered to the mammal and the SLC polypeptide is produced by a syngeneic mammalian cell that has been transduced with an expression vector encoding the SLC polypeptide. In a highly preferred embodiment, the cells are exposed to a SLC polypeptide that is expressed by a mammalian cell that has been transduced with an expression vector encoding the SLC polypeptide. A related embodiment of the invention consists of syngeneic host cells that have been transduced with an expression vector encoding the SLC polypeptide. In highly preferred embodiments of this aspect of the invention, the syngeneic host cells have been transduced with an expression vector encoding the SLC polypeptide in vivo.

Yet another embodiment of the invention is a method of inhibiting the growth of cancer cells (most preferably spontaneous cancer cells) in a mammal comprising administering secondary lymphoid tissue chemokine (SLC) to the mammal; wherein the SLC is administered to the mammal by transducing the cells of the mammal with a polynucleotide encoding the SLC shown in SEQ ID NO: 1 such that the transduced cells express the SLC polypeptide in an amount sufficient to inhibit the growth of the cancer cells. Preferably the vector is administered to a mammal via intratumoral injection, or alternatively via intra-lymph node injection.

Yet another embodiment of the invention is a method of inhibiting the growth of cancer cells (most preferably spontaneous cancer cells) in a mammal comprising administering secondary lymphoid tissue chemokine (SLC) ex vivo to the mammalian cells. As illustrated in Example 10, in a preferred embodiment, the SLC is administered to the mammal by transducing the cells of the mammal with a polynucleotide encoding SLC (e.g. SLC as shown in SEQ ID NO: 1) such that the transduced cells express the SLC polypeptide in an amount sufficient to inhibit the growth of syngeneic cancer cells. In such embodiments the population of cells can be removed from the mammal by any one of the variety of methods known in the art. Typically the cells are removed from the mammal at a site proximal to the cancer cells (e.g. at the site of the tumor or from a lymph node proximal to the tumor) and then reintroduced into the mammal after administration of the SLC (typically a site proximal to the cancer cells such as at the site of the tumor or at a lymph node proximal to the tumor).

One such embodiment of the invention is an ex vivo an method of treating a syngeneic cancer in a mammalian subject comprising administering a therapeutically effective amount of an SLC to the subject; wherein the SLC is expressed by a mammalian cell that has been transduced with an expression vector encoding the SLC polypeptide shown in SEQ ID NO: 1 or NO: 3, wherein the expression vector is administered after being transduced into a DC cell derived from the mammalian subject.

A related embodiment of the invention is an ex vivo method of treating a syngeneic cancer in a mammalian subject comprising administering a therapeutically effective amount of an SLC to the subject, wherein the SLC so administered is expressed by an autologous cell transduced with a polynucleotide encoding the SLC polypeptide of SEQ ID NO: 1; and further wherein the autologous cell is administered to the mammalian subject.

Yet another embodiment of the invention is an ex vivo method of facilitating in vivo tumor antigen uptake and presentation by an antigen presenting cell in a mammalian subject comprising transducing a syngeneic cell with a vector encoding the SLC polypeptide of SEQ ID NO: 1 so that the SLC polypeptide is expressed by the syngeneic cell and placing the syngeneic cell proximal to a syngeneic tumor cell expressing the tumor antigen. Preferably the syngeneic cell is an autologous DC cell, although the use of analogous antigen presenting cells known in the art is also contemplated.

Another embodiment of the invention is an method of attracting a T lymphocyte or a mature host dendritic cell to a site of a syngeneic tumor (e.g. an adenocarcinoma) in a mammal comprising the steps of: obtaining a dendritic cell from the mammal; introducing an exogenous polynucleotide encoding secondary lymphoid tissue chemokine as shown in SEQ ID NO: 1 into the dendritic cell (e.g. via transduction with a vector comprising this sequence) so that the cell expresses the secondary lymphoid tissue chemokine; and then placing the dendritic cell generated in this manner at the site of the syngeneic tumor in the mammal (e.g. via intratumoral injection); wherein the secondary lymphoid tissue chemokine expressed by the dendritic cell then attracts the T lymphocyte or the mature host dendritic cell to the site of the syngeneic tumor in the mammal via chemotaxis. As shown in Example 10, this method can be successfully employed to elicit a significant chemotaxis of peripheral blood lymphocytes and dendritic cells to the site of a tumor in vivo. Correspondingly, sixty percent of mice treated with method showed the complete eradication of syngeneic tumors treated with this method while only 12% of mice treated with unmodified or control dendritic cells responded. This method has a number of uses. For example this method can be applied to therapeutic contexts (e.g. in the treatment of individuals suffering from a cancer). In addition, this method provides a model for dissecting the various physiological process associated with immunosurveillance, in particular the natural ability that mammals have to respond to cancers. In addition, this model can be used to study the coordinate use of various known chemotherapeutic agents, for example the effect that a specific chemotherapeutic agent has on the immune response associated with the chemotaxis of peripheral blood lymphocytes and dendritic cells to the site of a tumor in vivo.

In such methods, the autologous cell expressing an endogenous SLC can be administered to the mammalian subject by a variety of methods known in the art. Preferably the autologous cell expressing an endogenous SLC is administered to the subject by intratumoral injection. Alternatively the autologous cell expressing an endogenous SLC is administered to the subject by intra-lymph node injection. Such methods can be used in the treatment of a variety of cancers, most preferably adenocarcinomas.

In alternative embodiments of the invention, SLC is administered as an SLC polypeptide in an amount sufficient to modulate a physiological process in the target cell (e.g. to upregulate the expression of polypeptides associated with immunosurveillance), wherein the physiological process so modulated facilitates the target cell's inhibition of the growth of syngeneic cancer cells.

Other embodiments of the invention include methods for the preparation of a medication for the treatment of pathological conditions including cancer by preparing a SLC composition for administration to a mammal having the pathological condition. A related method is the use of an effective amount of a SLC in the preparation of a medicament for the treatment of cancer, wherein the cancer cells are syngeneic cancer cells. Such methods typically involve the steps of including an amount of SLC sufficient to modulate a cytokine profile as discussed above and/or inhibit the growth of syngeneic (preferably spontaneous) cancer cells in vivo and an appropriate amount of a physiologically acceptable carrier. As is known in the art, optionally other agents can be included in these preparations.

Throughout this application, various publications are referenced (within parentheses for example). The disclosures of these publications are hereby incorporated by reference herein in their entireties. For example, certain general methods that are related to methods used with the invention disclosed herein are described in International Patent Application Number WO 00/38706, the contents of which are incorporated herein by reference. In order to facilitate an understanding of various typical aspects of the invention, certain aspects of these incorporated materials are reproduced herein.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention. However, the invention is only limited by the scope of the appended claims.

### EXAMPLES

### Example 1

### Methods and Materials for Examining Immunomodulatory Molecules Such as SLC in Syngeneic Transplantable Tumor Models

### 1. Cell Culture and Tumorigenesis Models

Two weakly immunogenic lung cancers, line 1 alveolar carcinoma (L1C2, H-2d) and Lewis lung carcinoma (3LL, H-2b), were utilized for assessment of antitumor responses in vivo. The cells were routinely cultured as monolayers in 25-cm³ tissue culture flasks containing RPMI 1640 (Irvine Scientific, Santa Ana, Calif.) supplemented with 10% FBS (Gemini Bioproducts, Calabasas, Calif.), penicillin (100 U/ml), streptomycin (0.1 mg/ml), 2 mM glutamine (JRH Biosciences, Lenexa, Kans.) and maintained at 37° C. in a humidified atmosphere containing 5% CO₂ in air. The cell lines were Mycoplasma free, and cells were utilized up to the tenth passage before thawing frozen stock cells from liquid N₂. For tumorigenesis experiments, 105 3LL or L1C2 tumor cells were inoculated by s.c. injection in the right suprascapular area of C57BL/6 or BALB/c mice, and tumor volume was monitored three times per week. Five-day-old established tumors were treated with intratumoral injection of 0.5 μg of murine recombinant SLC or PBS diluent (Pepro Tech, Rocky Hill, N.J.) administered three times per week for 2 weeks. The endotoxin level reported by the manufacturer was <0.1 ng/μg (1 EU/μg) of SLC. The amount of SLC (0.5 μg) used for injection was determined by the in vitro biological activity data provided by the manufacturer. Maximal chemotactic activity of SLC for total murine T cells was 100 ng/ml. For in vivo evaluation of SLC-mediated antitumor properties, we utilized 5-fold more than this amount for each intratumoral injection. Tumorigenesis experiments were also performed in which equivalent amounts of murine serum albumin were utilized (Sigma, St. Louis, Mo.) as an irrelevant protein for control injections. Experiments were also performed in which the SLC was administered at the time of tumor inoculation. To determine the importance of the immune system in mediating antitumor responses after SLC administration, tumorigenesis experiments were conducted in SCID beige CB17 mice. SLC was administered s.c. at the time of tumor inoculation and then three times per week. CD4 and CD8 knockout mice were utilized to determine the contribution of CD4 and CD8 cells in tumor eradication. Two bisecting diameters of each tumor were measured with calipers. The volume was calculated using the formula (0.4) (ab2), with a as the larger diameter and b as the smaller diameter.

2. Cytokine Determination from Tumor Nodules, Lymph Nodes, and Spleens

The cytokine profiles in tumors, lymph nodes, and spleens were determined in both SLC and diluent-treated mice as previously described (Sharma et al., J. Immunol. 163:5020). Non necrotic tumors were harvested, cut into small pieces, and passed through a sieve (Bellco Glass, Vineland, N.J.). Tumor-draining lymph nodes and spleens were harvested from SLC-treated tumor-bearing, control tumor-bearing, and normal control mice. Lymph nodes and spleens were teased apart, RBC depleted with double-distilled H2O, and brought to tonicity with 1×PBS. Tumor nodules were evaluated for the production of IL-10, IL-12, GM-CSF, IFN-γ, TGF-β, vascular endothelial growth factor (VEGF), monokine induced by IFN-γ (MIG), and IP-10 by ELISA and PGE2 by enzyme immunoassay (EIA) in the supernatants after an overnight culture. Tumor-derived cytokine and PGE2 concentrations were corrected for total protein by Bradford assay (Sigma, St. Louis, Mo.). For cytokine determinations after secondary stimulation with irradiated tumor cells (5×10 6 cells/ml), splenic or lymph node-derived lymphocytes were cocultured with irradiated 3LL (105 cells/ml) at a ratio of 50:1 in a total volume of 5 ml. After an overnight culture, supernatants were harvested and GM-CSF, IFN-γ, IL-12, and IL-10 determined by ELISA.

### 3. Cytokine ELISA

Cytokine protein concentrations from tumor nodules, lymph nodes and spleens were determined by ELISA as previously described (Huang et al., Cancer Res. 58:1208). Briefly, 96-well Costar (Cambridge, Mass.) plates were coated overnight with 4 μg/ml of the appropriate anti-mouse mAb to the cytokine being measured. The wells of the plate were blocked with 10% fetal bovine serum (Gemini Bioproducts) in PBS for 30 min. The plate was then incubated with the Ag for 1 h, and excess Ag was washed off with PBS-Tween. The plate was incubated with 2 μg/ml biotinylated mAb to the appropriate cytokine (PharMingen, San Diego, Calif.) for 30 min, and excess Ab was washed off with PBS-Tween. The plates were incubated with avidin peroxidase, and after incubation in OPD substrate to the desired extinction, the subsequent change in color was read at 490 nm with a Microplate Reader (Molecular Dynamics, Sunnyvale, Calif.). The recombinant cytokines used as standards in the assay were obtained from PharMingen. IL-12 (Biosource) and VEGF (Oncogene Research Products, Cambridge, Mass.) were determined by kits according to the manufacturer's instructions. MIG and IP-10 were quantified by a modification of a double ligand method as previously described (Standiford et al., J. Clin. Invest. 86:1945). The MIG and IP-10 Abs and protein were from R&D (Minneapolis, Minn.). The sensitivities of the IL-10, GM-CSF, IFN-γ, TGF-β, MIG, and IP-10 ELISA were 15 pg/ml. For IL-12 and VEGF, the sensitivities were 5 pg/ml.

### 4. PGE2 EIA

PGE2 concentrations were determined using a kit from Cayman Chemical (Ann Arbor, Mich.) according to the manufacturer's instructions as previously described (Huang et al., Cancer Res. 58:1208). The EIA plates were read by a Molecular Dynamics Microplate Reader.

### 5. Cytolytic Experiments

Cytolytic activity was assessed as previously described (Sharma et al., J. Immunol. 163:5020). To quantify tumor cytolysis after a secondary stimulation with irradiated tumor cells, lymph node-derived lymphocytes (5×10⁶ cells/ml) from SLC-treated and diluent tumor-bearing mice were cultured with irradiated 3LL (10⁵ cells/ml) tumors at a ratio of 50:1 in a total volume of 5 ml. After a 5-day culture, the lytic capacity of lymph node-derived lymphocytes were determined against chromium-labeled (⁵¹Cr, Amersham Arlington, Heights, Ill.; sp. act. 250-500 mCi/mg) 3LL targets at varying E:T ratios for 4 h in 96-well plates. Spontaneous release and maximum release with 5% Triton X also were assessed. After the 4-h incubation, supernatants were removed and activity was determined with a gamma counter (Beckman, Fullerton, Calif.). The percent specific lysis was calculated by the formula: % lysis=100×(experimental cpm−spontaneous release)/(maximum release−spontaneous release).

### 6. Flow Cytometry

For flow cytometric experiments, two or three fluorochromes (PE, FITC, and Tri-color) (PharMingen) were used to gate on the CD3 T lymphocyte population of tumor nodule single-cell suspensions. DCs were defined as the CD11c and DEC 205 bright populations within tumor nodules and lymph nodes. Cells were identified as lymphocytes or DC by gating based on forward and side scatter profiles. Flow cytometric analyses were performed on a FACScan flow cytometer (Becton Dickinson, San Jose, Calif.) in the University of California, Los Angeles, Jonsson Cancer Center Flow Cytometry Core Facility. Between 5,000 and 15,000 gated events were collected and analyzed using Cell Quest software (Becton Dickinson).

### 7. Intracellular Cytokine Analysis

T lymphocytes from single-cell suspensions of tumor nodules and lymph nodes of SLC-treated and diluent-treated 3LL tumor-bearing mice were depleted of RBC with distilled, deionized H₂O and were evaluated for the presence of intracytoplasmic GM-CSF and IFN-γ. Cell suspensions were treated with the protein transport inhibitor kit GolgiPlug (PharMingen) according to the manufacturer's instructions. Cells were harvested and washed twice in 2% FBS-PBS. Cells (5×10) cells were resuspended in 200 μl of 2% FBS-PBS with 0.5 μg FITC-conjugated mAb specific for cell surface Ags CD3, CD4, and CD8 for 30 min at 4° C. After two washes in 2% FBS-PBS, cells were fixed, permeabilized, and washed using the Cytofix/Cytoperm Kit (PharMingen) following the manufacturer's protocol. The cell pellet was resuspended in 100 μl Perm/Wash solution and stained with 0.25 μg PE-conjugated anti-GM-CSF and anti-IFN-γ mAb for intracellular staining. Cells were incubated at room temperature in the dark for 30 min, washed twice, resuspended in 300 μl PBS, 2% paraformaldehyde solution, and analyzed by flow cytometry.

### 8. Typical SLC Polypeptides.

Table 4 below provides illustrative human and murine SLC polypeptide sequences.

TABLE 4
Human SLC
(SEQ ID NO: 1)
MAQSLALSLLILVLAFGIPRTQGSDGGAQDCCLKYSQRKIPA
KVVRSYRKQEPSLGCSIPAILFLPRKRSQAELCADPKELWVQ
QLMQHLDKTPSPQKPAQGCRKDRGASKTGKKGKGSKGCKRTE
RSQTPKGP
Murine SLC
(SEQ ID NO: 2)
MAQMMTLSLLSLDLALCIPWTQGSDGGGQDCCLKYSQKKIPY
SIVRGYRKQEPSLGCPIPAILFLPRKHSKPELCANPEEGWVQ
NLMRRLDQPPAPGKQSPGCRKNRGTSKSGKKGKGSKGCKRTE
QTQPSRG

### Example 2

### Examining Immunomodulatory Molecules in Syngeneic Transplantable Tumor Models Using SLC as a Illustrative Molecule

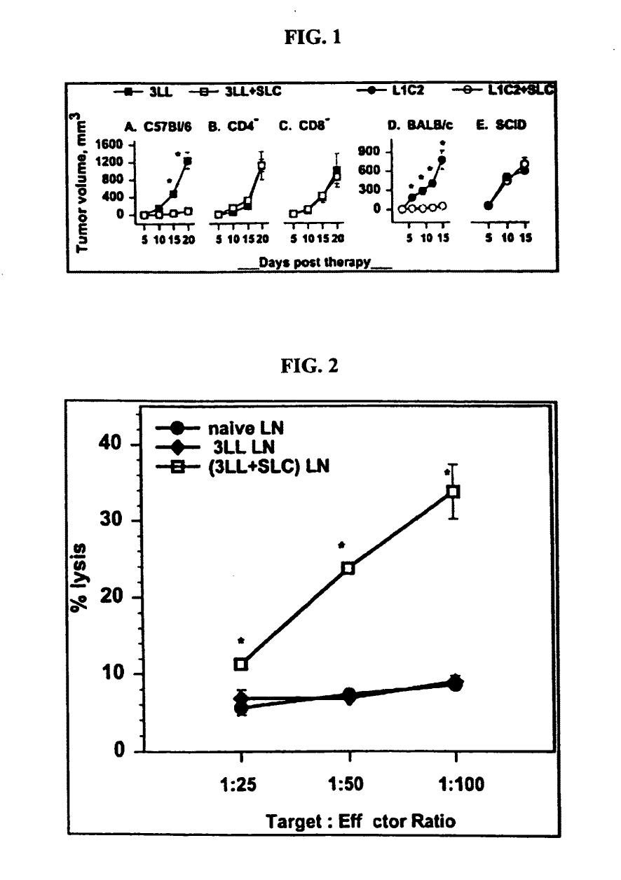

The disclosure provided herein tests antitumor properties of SLC utilizing two syngeneic transplanted murine lung cancer models. In both models, intratumoral SLC administration caused significant reduction in tumor volumes compared with diluent-treated tumor-bearing control mice (p<0.01), and 40% of mice showed complete tumor eradication (FIGS. 1, A and D). To determine whether the decrease in tumor volumes resulted from a direct effect of SLC on L1C2 and 3LL, the in vitro proliferation of the tumor cells was assessed in the presence of SLC. SLC (200 ng/ml) was added to 105 L1C2 and 3LL cells plated in 12-well Costar plates, and cell numbers were monitored daily for 3 days. SLC did not alter the in vitro proliferation rates of these tumor cells.

To evaluate the role of host immunity in SLC-mediated antitumor responses, SLC was injected intratumorally in tumor-bearing SCID beige CB17 mice. SLC administration did not alter tumor volumes in SCID mice (FIG. 1E). Similarly, in CD4 and CD8 knockout mice, SLC failed to reduce tumor volumes, indicating that SLC-mediated antitumor responses were both CD4 and CD8 dependent (FIG. 1, B and C).

Because tumor progression can be modified by host cytokine profiles (Alleva et al., J. Immunol. 153:1674; Rohrer et al., J. Immunol. 155:5719), the cytokine production from tumor nodules after intratumoral SLC administration was examined. The following cytokines were measured: VEGF, IL-10, PGE2, TGF-β, IFN-γ, GM-CSF, IL-12, MIG, and IP-10 (Table 1A). The production of these cytokines were evaluated for the following reasons. The tumor site has been documented to be an abundant source of PGE-2, VEGF, IL-10, and TGF-β, and the presence of these molecules at the tumor site have been shown to suppress immune responses (Huang et al., Cancer Res. 58:1208; Bellone et al., Am. J. Pathol. 155:537; Gabrilovich et al., Nat. Med. 2:1096). VEGF, PGE2, and TGF-β have also previously been documented to promote angiogenesis (Fajardo et al., Lab. Invest. 74:600; Ferrara et al., Breast Cancer Res. Treat. 36:127; 28; Tsujii et al., Cell 93:705). Abs to VEGF, TGF-β, PGE-2 and IL-10 have the capacity to suppress tumor growth in in vivo model systems. VEGF has also been shown to interfere with DC maturation (Gabrilovich et al., Nat. Med. 2:1096). Both IL-10 and TGFβ are immune inhibitory cytokines that may potently suppress Ag presentation and antagonize CTL generation and macrophage activities, thus enabling the tumor to escape immune detection (Sharma et al., J. Immunol. 163:5020; Bellone et al., Am. J. Pathol. 155:537). Compared with tumor nodules from diluent-treated tumor-bearing controls, mice treated intratumorally with SLC had significant reductions of PGE2 (3.5-fold), VEGF (4-fold), IL-10 (2-fold) and TGF-β (2.3-fold) (Table 1A). An overall decrease in IL-10 and TGFβ at the tumor site after SLC administration may have promoted Ag presentation and CTL generation. The decrease in VEGF and TGF-β at the tumor site after SLC administration may have contributed to an inhibition of angiogenesis. In contrast, there was a significant increase in IFN-γ (5-fold), GM-CSF (10-fold), IL-12 (2-fold), MIG (6.6-fold), and IP-10 (2-fold) after SLC administration (Table 1A).

Although IL-12 is a key inducer of type 1 cytokines, IFN-γ is a type 1 cytokine that promotes cell-mediated immunity. Increases in IL-12 (2-fold) could explain the relative increase in IFN-γ (5-fold) at the tumor site of SLC-treated mice (Table 1A). The tumor cells used for this study do not make detectable levels of IL-12. We therefore anticipate that macrophages and DC are the predominant sources of IL-12 at the tumor site.

MIG and IP-10 are potent angiostatic factors that are induced by IFN-γ and may be responsible, in part, for IL-12-mediated tumor reduction (Strieter et al., Biochem. Biophys. Res. Commun. 210:51; Tannenbaum et al., J. Immunol. 161:927; Arenberg et al., J. Exp. Med. 184:981). Hence, an increase in IFN-γ at the tumor site of SLC-treated mice could explain the relative increase in MIG (6.6-fold) and IP-10 (2-fold) (Table 1A). Both MIG and IP-10 are chemotactic for stimulated CXCR3-expressing T lymphocytes, and this could also increase IFN-γ at the tumor site (Farber et al., J. Leukocyte Biol. 61:246). An increase in GM-CSF (10-fold) in the tumor nodules of SLC treated mice could enhance DC maturation and Ag presentation (Banchereau et al., Nature 392:245).

Based on the current results, the decrease in immunosuppressive cytokines and concomitant increase in type 1 cytokines could be a direct effect of SLC on the cells resident within the tumor nodules. Alternatively, these changes could be a result of SLC-recruited T cells and DC. To begin to address this question, we evaluated the production of type 1 and immunosuppressive cytokines from tumor- and lymph node-derived cells in response to SLC in vitro. Tumor cells (1×10⁶) or lymph node-derived cells (5×10⁶) were cocultured with SLC (200 ng/ml) for 24 h for cytokine determinations. SLC did not affect tumor cell production of VEGF, TGF-β, IL-10, or PGE-2. Compared with the control untreated lymph node cells SLC significantly increased lymph node-derived IL-12 (288±15 pg/ml vs 400±7 pg/ml) while decreasing IL-10 (110±5 pg/ml vs 67±1 pg/ml), PGE2 (210±4 pg/ml vs 70±2 pg/ml), and TGF-β (258±9 pg/ml vs 158±7 pg/ml) production in an overnight in vitro culture. SLC did not alter lymph node-derived lymphocyte production of IFN-γ and GM-CSF in vitro. Because SLC is documented to have antiangiogenic effects (Soto et al., Proc. Natl. Acad. Sci. USA 95:8205; Arenberg et al., Am. J. Respir. Crit. Care Med. 159:A746), the tumor reductions observed in these models may be due to T cell-dependent immunity as well as a participation by T cells in inhibiting angiogenesis (Tannenbaum et al., J. Immunol. 161:927). Further studies will be necessary to delineate the cell types and proteins critical for the decrease in immunosuppressive cytokines and the increase in type 1 cytokines after SLC administration.

To determine whether the increase in GM-CSF and IFN-γ in the tumor nodules in response to SLC could be explained by an increase in the frequency of CD4 and CD8 T cell subsets secreting these cytokines, flow cytometric analyses were performed. CD3 T cells that stained positively for cell surface markers CD4 or CD8 were evaluated in single-cell suspensions from tumor nodules. In the tumor nodules of SLC-treated mice, within the gated T lymphocyte population, there was a significant increase in the frequency of CD4 and CD8 T lymphocytes in comparison to diluent-treated mice (25 and 33% vs 15 and 11%, respectively; p<0.01). The GM-CSF and IFN-γ profile of CD4 and CD8 T cells at the tumor sites and lymph nodes were determined by intracytoplasmic staining. SLC administration resulted in an increased frequency of CD4 and CD8 T lymphocytes from tumor nodules and lymph nodes secreting GM-CSF and IFN-γ (Table 2A).

DC are uniquely potent APC involved in the initiation of immune responses, and it is well documented that SLC strongly attracts mature DC (Chan et al., Blood 93:3610; Banchereau et al., Nature 392:245). Because intratumoral SLC administration led to significant tumor reduction, we questioned whether intratumoral SLC administration led to enhanced DC infiltration of tumor nodules and lymph nodes. Single-cell suspensions of tumor nodules and lymph nodes from SLC and diluent-treated tumor-bearing mice were stained for the DC surface markers CD11c and DEC205. In the SLC-treated tumor-bearing mice, there was an increase in both the frequency and mean channel fluorescence intensities of DC for cell surface staining of CD11c and DEC205 in the tumor nodules and lymph nodes in comparison with diluent-treated 3LL tumor-bearing mice (Table 2A). These findings indicate that intratumoral SLC administration effectively recruited DC to the tumor site

We next asked whether intratumoral SLC administration could induce significant systemic immune responses. To address this question, lymph node and splenocytes from SLC and diluent-treated tumor-bearing mice were cocultured with irradiated tumor cells for 24 h, and GM-CSF, IFN-γ, IL-10, and IL-12 levels were determined by ELISA. After secondary stimulation with irradiated tumor cells, splenocytes and lymph node-derived cells from SLC-treated tumor-bearing mice secreted significantly increased levels of IFN-γ (13- to 28-fold), GM-CSF (3-fold spleen only) and IL-12 (1.3- to 4-fold). In contrast, IL-10 secretion was reduced (6- to 9-fold) in SLC-treated mice (Table 3A). Moreover, intratumoral SLC administration led to enhanced lymph node-derived lymphocyte cytolytic activity against the parental tumor cells (FIG. 2). We speculate that the phenotype of the effector cell population in the cytolytic experiments is CD8+T lymphocytes because SLC did not affect tumor growth in SCID mice. However, tumorigenesis experiments utilizing CD4 and CD8 knockout mice demonstrate the importance of both CD4 and CD8 T lymphocytes subsets for effective tumor reduction. Because CD4 T lymphocytes can also act as cytolytic effectors (Sun et al., Cell. Immunol. 195:81; Semino et al., Cell. Immunol. 196:87), further studies will be required to delineate the role of CD4 T lymphocytes in SLC-mediated tumor reduction.

The results of this study indicate that intratumoral SLC administration leads to colocalization of both DC and T lymphocytes within tumor nodules and T cell dependent tumor rejection. These findings provide a strong rationale for further evaluation of SLC in tumor immunity and its use in cancer immunotherapy.

### Example 3

### Methods and Materials for Examining Immunomodulatory Molecules Such as SLC in Spontaneous Tumor Models

### 1. Cell Culture.

Clara cell lung tumor cells (CC-10 Tag and H-2q) were derived from freshly excised lung tumors that were propagated in RPMI 1640 (Irvine Scientific, Santa Ana, Calif.) supplemented with 10% FBS (GeminiBioproducts, Calabasas, Calif.), penicillin (100 units/ml), streptomycin (0.1 mg/ml), and 2 mM of glutamine (JRH Biosciences, Lenexa, Kans.) and maintained at 37° C. in humidified atmosphere containing 5% CO2 in air. After two in vivo passages, CC-10 TAg tumor clones were isolated. The cell lines were Mycoplasma free, and cells were used up to the tenth passage before thawing frozen stock cells from liquid N₂.

### 2. CC10TAg Mice.

The transgenic CC-10 TAg mice, in which the SV40large TAg is expressed under control of the murine Clara cell-specific promoter, were used in these studies (Magdaleno et al., Cell Growth Differ., 8: 145-155, 1997). All of the mice expressing the transgene developed diffuse bilateral bronchoalveolar carcinoma. Tumor was evident bilaterally by microscopic examination as early as 4 weeks of age. After 3 months of age, the bronchoalveolar pattern of tumor growth coalesced to form multiple bilateral tumor nodules. The CC-10 TAg transgenic mice had an average life span of 4 months. Extrathoracic metastases were not noted. Breeding pairs for these mice were generously provided by Francesco J. DeMayo (Baylor College of Medicine, Houston, Tex.). Transgenic mice were bred at the West Los Angeles Veteran Affairs vivarium and maintained in the animal research facility. Before each experiment using the CC-10 TAg transgenic mice, presence of the transgene was confirmed by PCR of mouse tail biopsies. The 5′ primer sequence was SM19-TAG: 5′-TGGACCTTCTAGGTCTTGAAAGG-3′ (SEQ ID NO: 3), and the 3′ primer sequence was SM36-TAG: 5′-AGGCATTCCACCACTGCTCCCATT-3′ (SEQ ID NO: 4). The size of the resulting PCR fragment is 650 bp. DNA (1 μg) was amplified in a total volume of 50 which contained 10 mM Tris-HCl (pH 8.3), 50 mM KCl, 200 μM each deoxynucleotidetriphosphates, 0.1 μM primers, 2.5 mM MgCl2, and 2.5 units of Taq polymerase. PCR was performed in a Perkin-Elmer DNA thermal cycler (Norwalk, Conn.). The amplification profile for the SV40 transgene consisted of 40 cycles, with the first cycle denaturation at 94° C. for 3 min, annealing at 58° C. for 1 min, and extension at 72° C. for 1 min, followed by 39 cycles with denaturation at 94° C. for 1 min, and the same annealing and extension conditions. The extension step for the last cycle was 10 min. After amplification, the products were visualized against molecular weight standards on a 1.5% agarose gel stained with ethidium bromide. All of the experiments used pathogen-free CC-10 TAg transgenic mice beginning at 4-5 week of age.

### 3. The SLC Therapeutic Model in CC-10 TAg Mice.

CC-10 TAg transgenic mice were injected in the axillary node region with murine recombinant SLC (0.5 μg/injection; Pepro Tech, Rocky Hill, N.J.) or normal saline diluent, which contained equivalent amounts of murine serum albumin (Sigma Chemical Co., St. Louis, Mo.) as an irrelevant protein for control injections. Beginning at 4-5 weeks of age, SLC or control injections were administered three times per week for 8 weeks. The endotoxin level reported by the manufacturer was <0.1 ng/μg (1 endotoxin unit/μg) of SLC. The dose of SLC (0.5 μg/injection) was chosen based on our previous studies (Arenberg et al., J. Exp. Med. 184:981) and the in vitro biological activity data provided by the manufacturer. Maximal chemotactic activity of SLC for total murine T cells was found to be 100 ng/ml. For in vivo evaluation of SLC-mediated antitumor properties we used 5-fold more than this amount for each injection. At 4 months, mice were sacrificed, and lungs were isolated for quantification of tumor surface area. Tumor burden was assessed by microscopic examination of H&E-stained sections with a calibrated graticule (a 1-cm² grid subdivided into 100 1-mm² squares). A grid square with tumor occupying >50% of its area was scored as positive, and the total number of positive squares was determined as described previously (Sharma et al., J. Immunol., 163: 5020-5028, 1999). Ten separate fields from four histological sections of the lungs were examined under high-power (×20 objective). Ten mice from each group were not sacrificed so that survival could be assessed.

4. Cytokine Determination from Tumor Nodules, Lymph Nodes, and Spleens.

The cytokine profiles in tumors, lymph nodes, and spleens were determined in both SLC and diluent-treated mice as described previously (Sharma et al., J. Immunol., 163: 5020-5028, 1999). Non-necrotic tumors were harvested and cut into small pieces and passed through a sieve (Bellco, Vineland, N.J.). Axillary lymph nodes and spleens were harvested from SLC-treated tumor-bearing, control tumor-bearing, and normal control mice. Lymph nodes and spleens were teased apart, RBC depleted with ddH2O, and brought to tonicity with 1×PBS. After a 24-h culture period, tumor nodule supernatants were evaluated for the production of IL-10, IL-12, GM-CSF, IFN-γ, TGF-β, VEGF, MIG, and IP-10 by ELISA and PGE-2 by EIA. Tumor-derived cytokine and PGE-2 concentrations were corrected for total protein by Bradford assay (Sigma Chemical Co.). For cytokine determinations after secondary stimulation with irradiated tumor cells, splenocytes (5×10⁶ cells/ml), were cocultured with irradiated (100 Gy, Cs¹³⁷ x-rays) CC-10 TAg tumor cells (10⁵ cells/ml) at a ratio of 50:1 in a total volume of 5 ml. After a 24-h culture, supernatants were harvested and GM-CSF, IFN-γ, and IL-10 determined by ELISA.

### 5. Cytokine ELISA.

Cytokine protein concentrations from tumor nodules, lymph nodes, and spleens were determined by ELISA as described previously (Sharma et al., Gene Ther., 4: 1361-1370, 1997). Briefly, 96-well Costar (Cambridge, Mass.) plates were coated overnight with 4 μg/ml of the appropriate antimouse mAb to the cytokine being measured. The wells of the plate were blocked with 10% FBS (Gemini Bioproducts) in PBS for 30 min. The plate was then incubated with the antigen for 1 h, and excess antigen was washed off with PBS/Tween 20. The plate was incubated with 2 μg/ml of biotinylated mAb to the appropriate cytokine (PharMingen) for 30 min, and excess antibody was washed off with PBS/Tween 20. The plates were incubated with avidin peroxidase, and after incubation in O-phenylenediamine substrate to the desired extinction, the subsequent change in color was read at 490 nm with a Molecular Devices Microplate Reader (Sunnyvale, Calif.). The recombinant cytokines used as standards in the assay were obtained from PharMingen. IL-12 (Biosource) and VEGF (Oncogene Research Products, Cambridge, Mass.) were determined using kits according to the manufacturer's instructions. MIG and IP-10 were quantified using a modification of a double ligand method as described previously (Standiford et al., J. Clin. Investig., 86: 1945-1953, 1990). The MIG and IP-10 antibodies and protein were obtained from R&D (Minneapolis, Minn.). The sensitivities of the IL-10, GM-CSF, IFN-γ, TGF-β, MIG, and IP-10 ELISA were 15 pg/ml. For IL-12 and VEGF the ELISA sensitivities were 5 pg/ml.

### 5. PGE2 EIA.

PGE2 concentrations were determined using a kit from Cayman Chemical Co. (Ann Arbor, Mich.) according to the manufacturer's instructions as described previously (Huang et al., Cancer Res., 58: 1208-1216, 1998). The EIA plates were read by a Molecular Devices Microplate reader (Sunnyvale, Calif.).

### 6. Flow Cytometry.

For flow cytometric experiments, two or three fluorochromes (PE, FITC, and Tri-color; PharMingen) were used to gate on the CD3T-lymphocyte population of tumor nodule, lymph node, and splenic single cell suspensions. DCs were defined as the CD11c and DEC 205 bright populations within tumor nodules, lymph nodes, and spleens. Cells were identified as lymphocytes or DCs by gating based on forward and side scatter profiles. Flow cytometric analyses were performed on a FACScan flow cytometer (Becton Dickinson, San Jose, Calif.) in the University of California, Los Angeles, Jonsson Cancer Center Flow Cytometry Core Facility. Between 5,000 and 15,000 gated events were collected and analyzed using Cell Quest software (Becton Dickinson).

### 7. Intracellular Cytokine Analysis.

T lymphocytes from single cell suspensions of tumor nodules, lymph nodes, and spleens of SLC-treated and diluent treated CC-10 TAg transgenic mice were depleted of RBC with distilled, deionized H₂O and were evaluated for the presence of intracytoplasmic GM-CSF and IFN-γ Cell suspensions were treated with the protein transport inhibitor kit Golgi Plug (PharMingen) according to the manufacturer's instructions. Cells were harvested and washed twice in 2% FBS/PBS. Cells (5×10⁵) were resuspended in 200 μl of 2% FBS/PBS with 0.5 μg of FITC-conjugated mAb specific for cell surface antigens CD3, CD4, and CD8 for 30 min at 4° C. After two washes in 2% FBS/PBS, cells were fixed, permeabilized, and washed using the Cytofix/Cytoperm kit (PharMingen) following the manufacturer's protocol. The cell pellet was resuspended in 100 μl of Perm/Wash solution and stained with 0.25 μg of PE-conjugated anti-GM-CSF and anti-IFN-γ mAb for intracellular staining. Cells were incubated at room temperature in the dark for 30 min and washed twice, resuspended in 300 μl of PBS/2% paraformaldehyde solution, and analyzed by flow cytometry.

### Example 4

### SLC Mediates Potent Antitumor Responses in a Murine Model of Spontaneous Bronchoalveolar Carcinoma

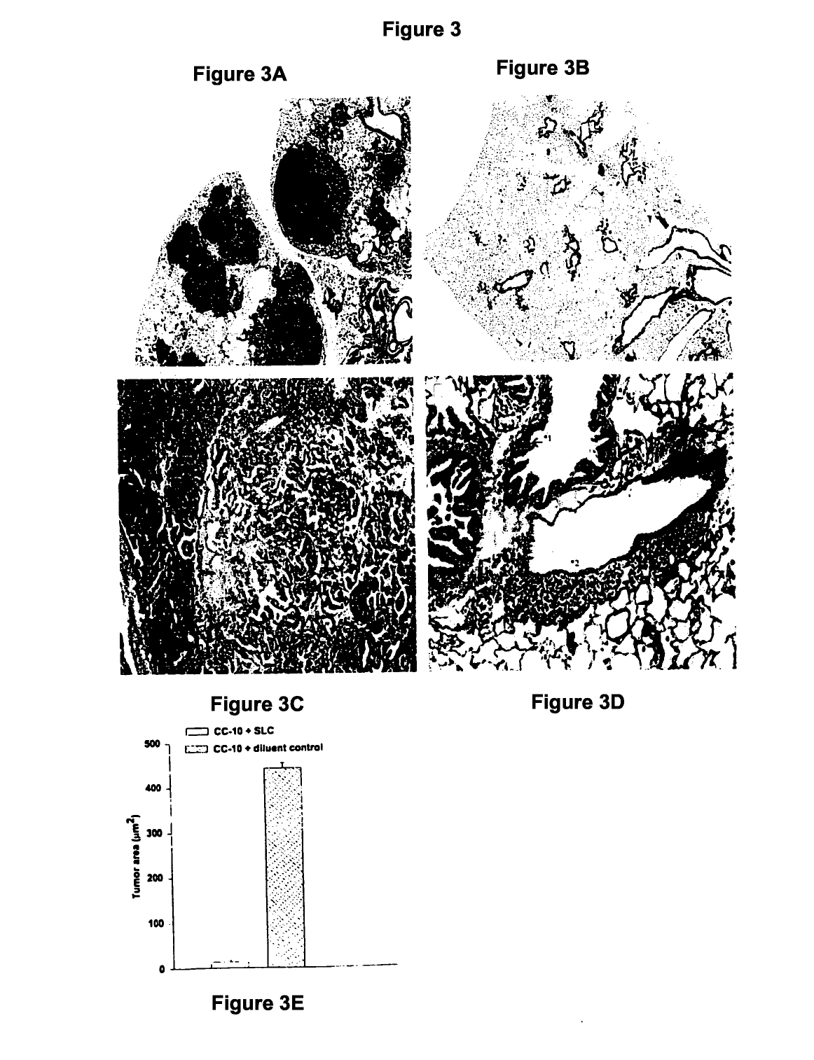

Using the material and methods described in Example 3, the antitumor efficacy of SLC in a spontaneous bronchoalveolar cell carcinoma model in transgenic mice in which the SV40 large TAg is expressed under control of the murine Clara cell-specific promoter, CC-10 was evaluated. (Magdaleno et al., Cell Growth Differ., 8: 145-155, 1997). Mice expressing the transgene develop diffuse bilateral bronchoalveolar carcinoma and have an average life span of 4 months. SLC (0.5 μg/injection) or the same concentration of murine serum albumin was injected in the axillary lymph node region beginning at 4 weeks of age, three times per week and continuing for 8 weeks. At 4 months when the control mice started to succumb because of progressive lung tumor growth, mice were sacrificed in all of the treatment groups, and lungs were isolated and paraffin embedded. H&E staining of paraffin-embedded lung tumor sections from control-treated mice revealed large tumor masses throughout both lungs with minimal lymphocytic infiltration (FIG. 3 A and C). In contrast, SLC-treated mice had significantly smaller tumor nodules with extensive lymphocytic infiltration (FIG. 3, B and D). Mice treated with SLC had a marked reduction in pulmonary tumor burden as compared with diluent treated control mice (FIG. 3E). SLC-treated mice had prolonged survival compared with mice receiving control injections. Median survival was 18±2 weeks for control-treated mice, whereas mice treated with SLC had a median survival of 34±3 weeks (P<0.001).

### Example 5

### SLC Treatment of Cc-10 Tag Mice Promotes Type 1 Cytokine and Antiangiogenic Chemokine Release and a Decline in the Immunosuppressive Cytokines TGF-β and VEGF

On the basis of previous reports indicating that tumor progression can be modified by host cytokine profiles (Alleva et al., J. Immunol., 153: 1674-1686, 1994; Rohrer et al., J. Immunol., 155: 5719-5727, 1995), we evaluated the cytokine production from tumor sites, lymph nodes, and spleen after SLC therapy. Cytokine profiles in the lungs, spleens, and lymph nodes of CC-10 TAg mice treated with recombinant SLC were compared with those in diluent-treated control mice bearing tumors as well as nontumor bearing controls. SLC treatment of CC-10 TAg mice led to systemic induction of Type 1 cytokines but decreased production of immunosuppressive mediators. Lungs, lymph node, and spleens were harvested, and after a 24-h culture period, supernatants were evaluated for the presence of VEGF, IL-10, IFN-γ, GM-CSF, IL-12, MIG, IP-10, and TGF-β by ELISA and for PGE-2 by EIA. Compared with lungs from the diluent-treated group, CC-10 TAg mice treated with SLC had significant reductions in VEGF (3.5-fold) and TGF-β (1.83-fold) but an increase in IFN-γ (160.5-fold), IP-10 (1.7-fold), IL-12 (2.1-fold), MIG (2.1-fold), and GM-CSF (8.3-fold; Table 1B). Compared with the diluent treated group, splenocytes from SLC-treated CC-10 TAg mice revealed reduced levels of PGE-2 (14.6-fold) and VEGF (20.5-fold) but an increase in GM-CSF (2.4-fold), IL-12 (2-fold), MIG (3.4-fold), and IP-10 (4.1-fold; Table 1B). Compared with diluent treated CC-10 TAg mice, lymph node-derived cells from SLC treated mice secreted significantly enhanced levels of IFN-γ (2.2-fold), IP-10 (2.3-fold), MIG (2.3-fold), and IL-12 (2.5-fold) but decreased levels of TGF-β (1.8-fold; Table 1B). The immunosuppressive mediators PGE-2 and IL-10 were not altered at the tumor sites of SLC-treated mice; however, there was a significant reduction in the level of PGE-2 in the spleen of SLC-treated mice. To determine whether SLC administration induced significant specific systemic immune responses, splenocytes from SLC and diluent treated CC-10 TAg mice were cocultured in vitro with irradiated CC-10 TAg tumor cells for 24 h, and GM-CSF, IFN-γ, and IL-10 were determined by ELISA. After secondary stimulation with irradiated tumor cells, splenocytes from SLC-treated tumor-bearing mice secreted significantly increased levels of IFN-γ (5.9-fold) and GM-CSF (2.2-fold). In contrast, IL-10 secretion was reduced 5-fold (Table 3B).

### Example 6

### SLC Treatment of CC-10 Tag Mice Leads to Enhanced DC and T-Cell Infiltrations of Tumor Sites, Lymph Nodes, and Spleen

To determine the cellular source of GM-CSF and IFN-γ, single cell suspensions of tumors, lymph nodes, and spleens were isolated from SLC and diluent control-treated CC-10 TAg mice. T-lymphocyte infiltration and intracellular cytokine production were assessed by flow cytometry. The cells were also stained to quantify DC infiltration at each site. Compared with the diluent-treated control group, the SLC-treated CC-10 TAg mice showed significant increases in the frequency of cells expressing the DC surface markers CD11c and DEC 205 at the tumor site, lymph nodes, and spleen (Table 2B). Similarly, as compared with the diluent-treated control group, there were significant increases in the frequency of CD4 and D8 cells expressing IFN-γ and GM-CSF at the tumor sites, lymph nodes, and spleen of SLC-treated CC-10 TAg mice (Table 2B).

### Example 7

### SLC-Mediated Anti-Tumor Responses Require IFN-γ, MIG and IP-10

Studies presented herein teach that the SLC-mediated anti-tumor response is accompanied by the enhanced elaboration of IFN-γ, IP-10 and MIG at the tumor site. IP-10, MIG and IFN-γ are known to have potent anti-tumor activities in vivo. In this context a study was undertaken to determine if the augmentation of these cytokines served as effector molecules in SLC mediated tumor reduction. Here we show that SLC-mediated anti-tumor responses require the cytokines IP-10, MIG and IFN-γ.

We determined the roles of IFN-γ, IFN-γ inducible protein IP-10 (IP-10) and monokine-induced by IFN-γ (MIG) in the in vivo SLC-mediated anti-tumor responses. Depletion of IP-10, MIG and IFN-γ in vivo significantly reduced the antitumor efficacy of SLC. Assessment of cytokine production at the tumor site showed an interdependence of IFN-γ, MIG and IP-10; neutralization of any one of these cytokines in vivo caused a concomitant decrease in all three cytokines. These findings indicate that the SLC-mediated anti-tumor response requires the induction of IP-10, MIG and IFN-γ at the tumor site.

### Materials and Methods

Cell Culture and Tumorigenesis Model

A weakly immunogenic lung cancer, Lewis lung carcinoma (3LL, H-2^{b}) was utilized for assessment of cytokines important for SLC-mediated anti-tumor responses in vivo. The cells were routinely cultured as monolayers in 25 cm³ tissue culture flasks containing RPMI 1640 medium (Irvine Scientific, Santa Anna, Calif.) supplemented with 10% fetal bovine serum (FBS) (Gemini Bioproducts, Calabasas, Calif.), penicillin (100 U/ml), streptomycin (0.1 mg/ml), 2 mM glutamine (JRH Biosciences, Lenexa, Kans.) and maintained at 37° C. in humidified atmosphere containing 5% CO₂ in air. The cell lines were mycoplasma free and cells were utilized up to the tenth passage before thawing frozen stock cells from liquid N₂. For tumorigenesis experiments, 10⁵ 3LL tumor cells were inoculated by s.c. injection in the right supra scapular area of C57Bl/6 and tumor volume was monitored 3 times per week. Five day established tumors were treated with intratumoral injection of 0.5 μg of murine recombinant SLC or PBS diluent (Pepro Tech, Rocky Hill, N.J.) administered three times per week for two weeks. The endotoxin level reported by the manufacturer was less than 0.1 ng per μg (1 EU/μg) of SLC. The amount of SLC (0.5 μg) used for injection was determined by the in vitro biological activity data provided by the manufacturer. Maximal chemotactic activity of SLC for total murine T cells was found to be 100 ng/ml. For in vivo evaluation of SLC-mediated anti-tumor properties we utilized 5 fold more than this amount for each intratumoral injection. Tumorigenesis experiments were also performed in which equivalent amounts of murine serum albumin were utilized (Sigma, St. Louis, Mo.) as an irrelevant protein for control injections. 24 hours prior to SLC treatment, and then three times a week, mice were treated i.p. with 35 mg/dose of anti-IP-10 or anti-MIG, and 100 μg/dose of purified IFN-γ (ATCC R4562) or 35 mg/dose of control antibody for the duration of the experiment. At doses of antibody administered there was a significant in vivo depletion of the respective cytokines at the tumor site. Two bisecting diameters of each tumor were measured with calipers. The volume was calculated using the formula (0.4) (ab²), with “a” as the larger diameter and “b” as the smaller diameter.

### Cytokine ELISA

MIG, IP-10 and IFN-γ were quantified using a modification of a double ligand method as previously described. The MIG and IP10 antibodies and recombinant cytokine proteins were from R&D (Minneapolis, Minn.). The IFN-γ antibodies pairs and recombinant cytokine were from Pharmingen. The sensitivities of the IFNγ, MIG and IP-10 ELISA were 15 pg/ml.

### Results

Because SLC is documented to have direct anti-angiogenic effects, the tumor reductions observed in our model could have been due to T cell-dependent immunity as well as participation by T cells secreting IFN-γ in inhibiting angiogenesis. IFN-γ mediates a range of biological effects that facilitate anticancer immunity. MIG and IP-10 are potent angiostatic factors that are induced by IFN-γ and hence we postulated that in addition to IFN-γ they are be responsible in part for the tumor reduction following SLC administration.

To determine if the co-localization of DC and T cells to the tumor site was sufficient for SLC-mediated anti-tumor responses and/or whether the accompanying relative increases in the cytokines MIG, IP-10 and IFN-γ at the tumor site play a role in tumor reduction, these cytokines were depleted with antibodies in SLC treated mice. Anti-IP-10, MIG and IFN-γ antibodies significantly inhibited the efficacy of SLC (* p<0.01 compared to the control antibody group). Cytokine determinations at the tumor site showed that the relative increase in MIG and IP-10 at the tumor site are IFN-γ dependent because neutralization of IFN-γ caused a decrease in these cytokines. Thus, an increase in IFN-γ at the tumor site of SLC-treated mice could explain the relative increases in IP-10 and MIG. The converse was also observed; IFN-γ production at the tumor site was found to be dependent on MIG and IP-10 because neutralization of these cytokines caused a decrease in IFN-γ. Thus IFN-γ, MIG and IP-10 in SLC treated mice showed an interdependence since in vivo neutralization of any one of these cytokines caused a concomitant decrease in all three cytokines. Both MIG and IP-10 are chemotactic for stimulated CXCR3-expressing activated T lymphocytes that could further amplify IFN-γ at the tumor site. Our results suggest that the anti-tumor properties of SLC may be due to its chemotactic capacity in colocalization of DC and T cells as well as the induction of key cytokines such as IFN-γ, IP-10, MIG.

10⁵ 3 LL tumors were implanted in C57Bl/6 mice. 5 days following tumor implantation, mice were treated intratumorally with 0.5 μg of recombinant murine SLC three times per week. One day before SLC administration, mice were given the respective cytokine antibody by i.p. injection. The antibodies were administered three times per week. SLC treated mice had a significant induction in IFN-γ, MIG and IP-10 compared to diluent treated control tumor bearing mice (p<0.001). Whereas neutralization of IFN-γ in vivo reduced IFN-γ, IP-10 and MIG, neutralization of MIG and IP-10 led to a relative decrease in those cytokines. Neutralization of MIG also led to a decrease in IFN-γ and IP-10. Results are expressed as pg/mg of total protein. Total protein was determined by the Bradford assay. Results of these experiments are provided in Table 5 below.

TABLE 5

| Treatment groups | IFNγ | MIG | IP10 |
|---|---|---|---|
| Diluent treated | 306 ± 25 | 599 ± 29 | 562 ± 54 |
| Control Ab + SLC | 2,200 ± 57 | 10,350 ± 159 | 10,900 ± 168 |
| Anti IFN + SLC | 800 ± 38 | 730 ± 27 | 5400 ± 14 |
| Anti IP-10 + SLC | 990 ± 102 | 3390 ± 150 | 2001 ± 45 |
| Anti MIG + SLC | 725 ± 33 | 7970 ± 138 | 5760 ± 78 |

### Example 8

### SLC-Mediated Anti-Tumor Responses in a Murine Model of a Gene Therapy-Based Approach

The data provided in the Examples above demonstrate how SLC polypeptide mediates syngeneic T Cell-dependent antitumor responses in vivo. To explore a gene therapy-based anti-tumor approach using a direct injectable vector, we made an adenoviral construct expressing murine SLC cDNA (Ad-SLC). In these constructs the cDNA for murine secondary lymphoid chemokine was cloned downstream of the CMV promoter in the Invitrogen pMH4 plasmid and was used as the shuttle vector.

The pJM17 plasmid that contains the entire E1-deleted Ad-5 genome was used as the recombination vector (for illustrative methods see, e.g., Cancer Gene Ther 1997 January-February; 4(1):17-25). Murine AdSLC was prepared through an in vitro recombination event in 293 cells through a recombination event between the shuttle plasmid pMH4 containing the murine SLC cDNA and the pJM17 plasmid.

Clones of Ad SLC were obtained by limiting dilution analysis of the ability of media to induce cytopathic effect on 293 cells and confirmed by murine SLC specific ELISA that we developed in our laboratory. Viral stocks were obtained by amplification of the 293 cells followed by CsCl purification, dialysis and storage as a glycerol (10% vol/vol) stock at −80° C. (see, e.g., Cancer Gene Ther 1997 January-February; 4(1):17-25).

In vitro transduction of Line 1 alveolar carcinoma cells (L1C2) and the Lewis Lung carcinoma cells (3LL, derived from C57BL/6) led to the production of 10 ng/ml/10⁶ cells/24 hr SLC by these cell lines at an MOI of 100:1 as determined by SLC specific ELISA. We next determined the in vivo antitumor efficacy of the Ad-SLC construct using the transplantable murine L1C2 lung tumor model. 10⁸ pfu of the viral stock was added to 100 of PBS for intratumoral injection into C57BL/6 mice. 10⁵ cells were injected in the right supra scapular region of the mice and 5 days later, the tumors treated with an intratumoral injection of Ad-SLC or control Ad vector once a week for three weeks at pfu's ranging from 10⁷-10⁹. The virus was injected into the tumor using an insulin syringe with the injectate was delivered slowly to allow for an even distribution of the virus particles in the tumor.

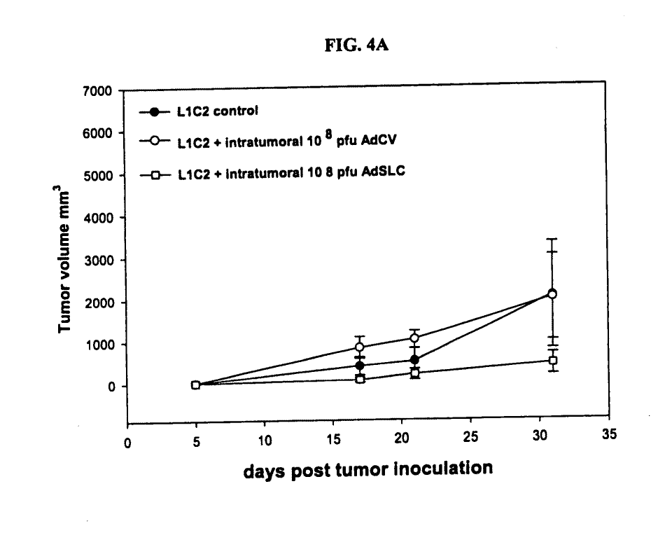

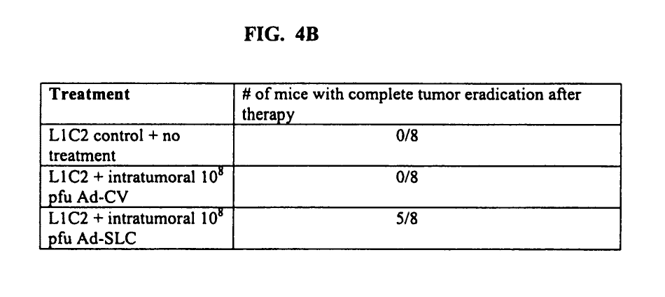

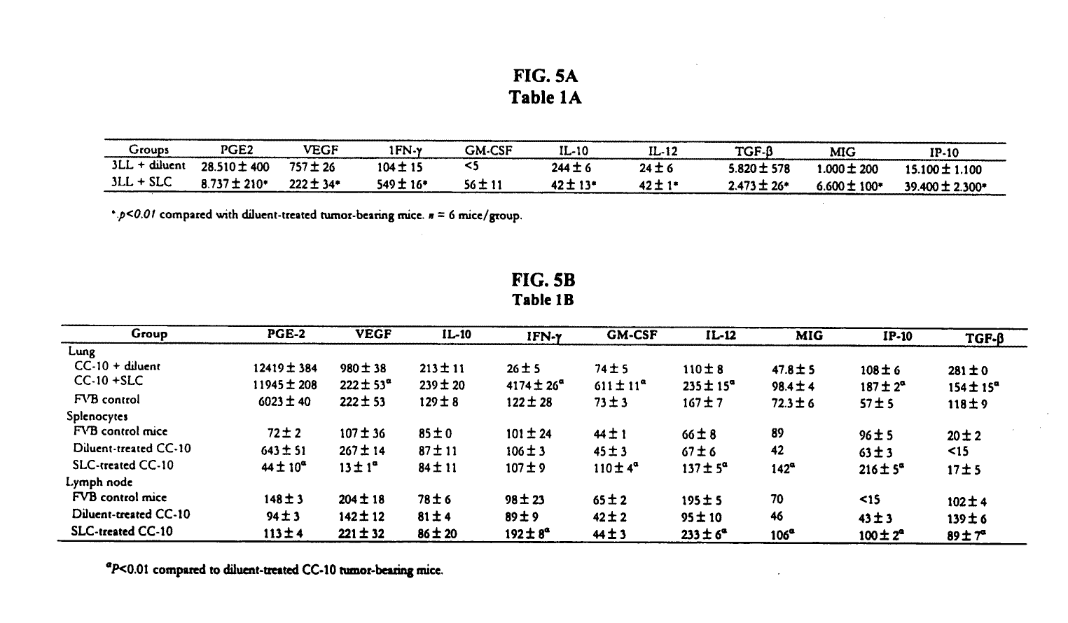

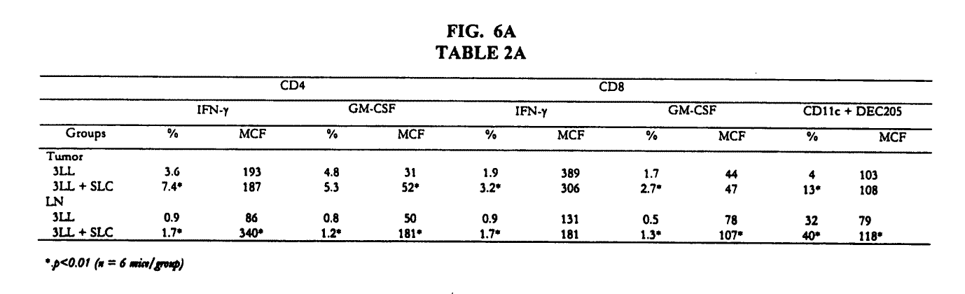

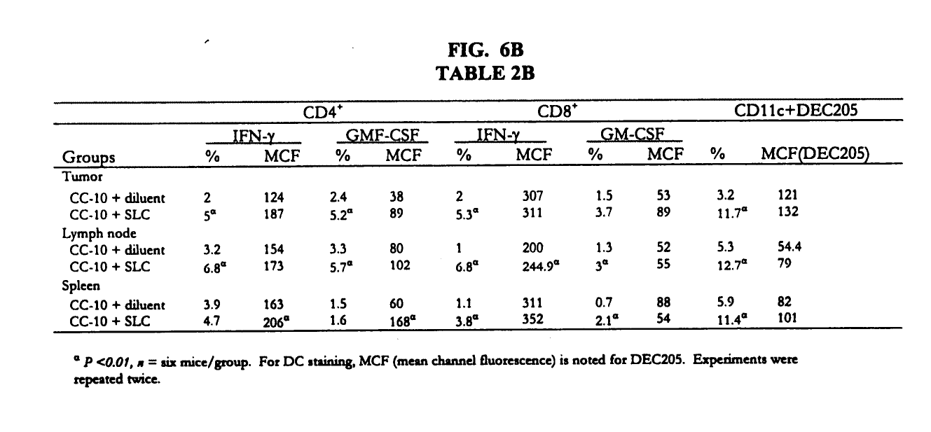

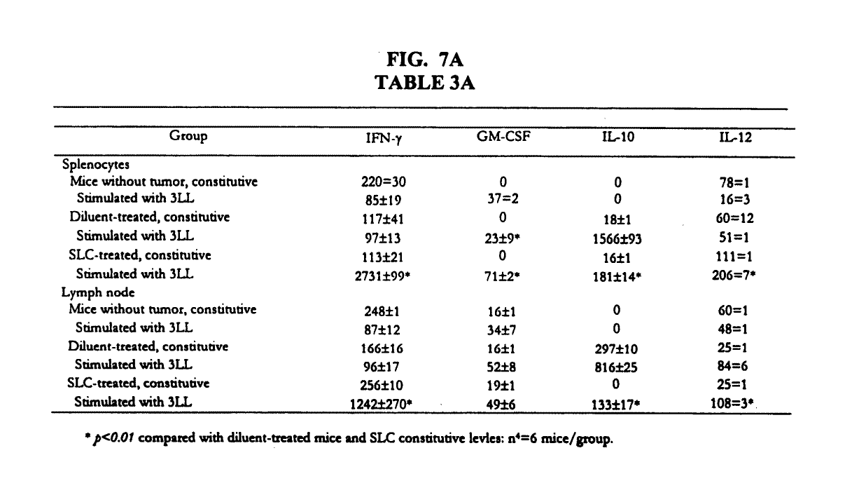

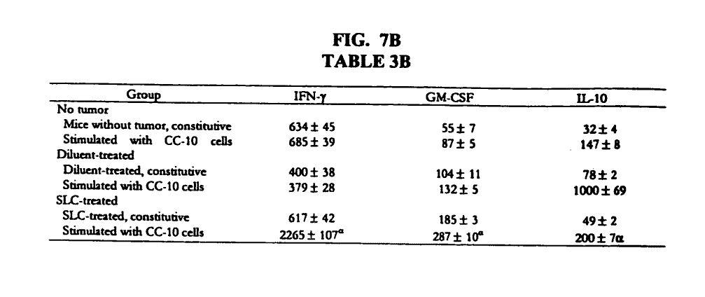

As illustrated in FIG. 4, intratumoral injection of the Ad-SLC vector led to the complete regression of the tumors in 60% of the mice whereas the control Ad vector did not have this effect. We also determined the antitumor efficacy of a single intratumoral dose of Ad-SLC at 10⁸ pfu and found it to be as effective as three doses. Mice rejecting their tumors in response to Ad-SLC therapy were able to reject a secondary challenge of 5×10⁵ parental tumors. These results indicate that an in vivo SLC gene therapy strategy can lead to significant tumor reduction in syngeneic lung cancer models.

The in vivo gene transfer methods disclosed herein provide clinically relevant models for treating cancers. In particular, these in vivo models are directly relevant cancer models because the cancer arise in a spontaneous manner (and are therefore syngeneic). In addition, the gene therapy methods disclosed herein directly parallel the clinical model, that is the administration of a polynucleotide encoding SLC polypeptide. The fact that the administration of this gene therapy vector is shown to reduce tumor burden provides direct evidence which strongly supports the use of such vectors in clinical methods for treating cancer. Consequently this model provides a particularly useful tool for optimizing and characterizing SLC based gene therapies.

### Example 9

### SLC-Mediated Anti-Tumor Responses in a Human Gene Therapy-Based Approach

A human gene therapy-based anti-tumor approach can be employed using a vector such as an adenoviral construct that expresses human SLC cDNA. In these constructs the cDNA for human secondary lymphoid chemokine can be cloned downstream of a promoter that allows an appropriate degree of expression such as a CMV promoter.

A plasmid such as the pJM17 plasmid that contains the entire E1-deleted Ad-5 genome can be used as the recombination vector (for illustrative methods see, e.g., Cancer Gene Ther 1997 January-February; 4(1):17-25). Human AdSLC can be prepared through an in vitro recombination event in 293 cells through a recombination event between a shuttle plasmid containing the human SLC cDNA and the recombination plasmid.

Clones of Ad SLC can be obtained by limiting dilution analysis of the ability of media to induce cytopathic effect on cells such as 293 cells and confirmed by human SLC specific ELISA that we developed in our laboratory. Viral stocks can be obtained by amplification of the cells followed by CsCl purification, dialysis and storage as a glycerol (10% vol/vol) stock at −80° C. (see, e.g., Cancer Gene Ther 1997 January-February; 4(1):17-25).

In vitro transduction of lines such as Line 1 alveolar carcinoma cells (L1C2) and the Lewis Lung carcinoma cells (3LL) can be used in the production of SLC by these cell lines at an MOI of 100:1 as determined by SLC specific ELISA. One can determine the in vivo antitumor efficacy of the ASLC construct using cells equivalent to the transplantable murine L1C2 lung tumor model. 10⁸ pfu of the viral stock can be added to 100 μl of PBS for intratumoral injection. 10⁵ cells can be injected in a region proximal to the tumor and 5 days later, the tumors can be treated with an intratumoral injection of SLC vector once a week for three weeks at pfu's ranging from 10⁷-10⁹. In one method, the virus can be injected into the tumor using an insulin syringe with the injectate can be delivered slowly to allow for an even distribution of the virus particles in the tumor.

### Example 10

### Use of Adenoviral CCL-21/SLC in Typical Intratumoral Dendritic Cell (Dc) Based Ex-Vivo Therapies in NSCLC

In this example we demonstrate a method for achieving in-situ tumor antigen uptake and presentation utilizing intratumoral administration of ex vivo-generated gene modified DC. In this example, in order to attract mature host DC to the tumor site, the DCs were transduced with an adenoviral vector construct expressing CCL-21 (secondary lymphoid tissue chemokine (SLC)). Because CCL-21 potently attracts mature DC and activated T cells, the intratumoral injection (i.t.) of DC expressing CCL-21 leads to potent antitumor responses in lung cancer models.

In this illustrative model, 10⁵ Line 1 alveolar cell carcinoma (L1C2) cells were utilized to establish subcutaneous tumors in syngeneic BALB/c mice. Established tumors were treated i.t. with 10⁶ DC-Ad-CCL-21 (10 ng/ml/10⁶ cells/24 hrs of CCL-21) at weekly intervals for 3 weeks. Sixty percent of the mice treated with DC-Ad-CCL-21 i.t. showed complete tumor eradication. In contrast only 12% of the mice treated with unmodified or control vector modified DCs (DC-Ad-CV) responded. Based on these results we constructed and characterized an adenoviral vector that expresses human CCL-21 (Ad-CCL-21). Human monocyte derived DCs were cultured in medium containing GM-CSF and IL-4. Following transduction on day 6, CCL-21 protein production was assessed on day 8 by ELISA. DCs transduced with Ad-CCL-21 at MOIs of 50:1 or 100:1 produced 71±15 ng/ml and 91±5 ng/ml/10⁶ cells/48 hours. At the MOIs evaluated, DC maintained cell viability as well as their immature phenotype without significantly upregulating CD83 or CCR7R expression. In addition as few as 10⁵ DC-Ad-CCL-21 caused significant chemotaxis of peripheral blood lymphocytes and LPS-stimulated DC.

These studies provide evidence for the successful use of intratumoral DC-Ad-CCL-21 therapy in NSCLC.

What is claimed is:

1. A method of effecting an increase in the expression of Interferon-γ (IFN-γ) polypeptides and a decrease in the expression of Transforming Growth Factor-β (TGF-β) polypeptides in a population of syngeneic mammalian cells including CD8 positive T cells, CD4 positive T cells, Antigen Presenting Cells and tumor cells comprising exposing the population of cells to an amount of secondary lymphoid tissue chemokine (SLC) polypeptide sufficient to inhibit the growth of the tumor cells.

2. A method as in claim 1, wherein the increase in the expression of Interferon-γ (IFN-γ) polypeptides is at least about two-fold and a decrease in the expression of Transforming Growth Factor-β (TGF-β) polypeptides is at least about two-fold as measured by an enzyme linked immunoadsorbent (ELISA) assay.

3. A method as in claim 1, wherein the inhibition of the growth of the syngeneic tumor cells is measured by quantification of tumor surface area.

4. A method as in claim 1, wherein the syngeneic tumor cells are spontaneous cancer cells.

5. A method as in claim 1, wherein the SLC is further used to effect an increase in Granulocyte-Macrophage colony stimulating factor (GM-CSF) polypeptides, monokine induced by IFN-γ (MIG) polypeptides, Interleukin-12 (IL-12) polypeptides or IFN-γ inducible protein 10 polypeptides; or to effect a decrease in Prostaglandin E(2) polypeptides or vascular endothelial growth factor (VEGF) polypeptides.

6. A method as in claim 1, wherein the syngeneic cells are exposed to SLC polypeptide administered to a mammal by intratumoral injection.

7. A method as in claim 1, wherein the syngeneic cells are exposed to SLC polypeptide administered to a mammal by intra-lymph node injection.

8. A method as in claim 1, wherein the SLC polypeptide is produced by a syngeneic mammalian cell that has been transduced with an expression vector encoding the SLC polypeptide.

9. A method as in claim 1, wherein the method further includes exposing the population of cells to a small molecule or polypeptide agent and observing the agent's effect on the expression of IFN-γ polypeptides or the expression of TGF-β polypeptides.

10. A method of attracting T lymphocyte or mature host dendritic cells to a site of a syngeneic tumor in a mammal, wherein the tumor developed spontaneously in vivo in the mammal, the method comprising the steps of:
(a) obtaining dendritic cells from the mammal;
(b) introducing an exogenous polynucleotide encoding a secondary lymphoid tissue chemokine comprising the amino acid sequence of SEQ ID NO: 1 into the dendritic cells so that the cells express the secondary lymphoid tissue chemokine; and
(c) placing dendritic cells generated in step (b) at the site of the syngeneic tumor in the mammal;
wherein the secondary lymphoid tissue chemokine expressed by the dendritic cells generated in step (b) attracts T lymphocyte or mature host dendritic cells to the site of the syngeneic tumor in the mammal.

11. The method of claim 10, wherein the syngeneic tumor is an adenocarcinoma.

12. The method of claim 10, wherein the SLC is expressed by dendritic cells transduced with an expression vector encoding the SLC polypeptide.

13. The method of claim 12, wherein the expression vector is administered in a composition that facilitates the transduction of cells proximal to the site of administration.

14. The method of claim 13, wherein the composition comprises a liposome.

15. The method of claim 10, wherein the SLC is expressed by dendritic cells infected with an adenovirus comprising a polynucleotide encoding the SLC polypeptide.

16. A method of inhibiting the growth of cancer cells in a mammal comprising administering secondary lymphoid tissue chemokine (SLC) to the mammal; wherein the SLC is administered to the mammal by transducing the cells of the mammal with a vector having a polynucleotide encoding the SLC shown in SEQ ID NO: 1 so that the transduced cells express the SLC polypeptide in an amount sufficient to inhibit the growth of the cancer cells.

17. The method of claim 16, wherein the vector having a polynucleotide encoding the SLC shown in SEQ ID NO: 1 is administered to the mammal by intratumoral injection.

18. The method of claim 16, wherein the vector having a polynucleotide encoding the SLC shown in SEQ ID NO: 1 is administered to the mammal by intra-lymph node injection.

19. The method of claim 16, wherein the syngeneic tumor cells are spontaneous cancer cells.

20. The method of claim 19, the syngeneic tumor is an adenocarcinoma.


SEQUENCE LISTING (4 sequences)

SEQ ID NO: 1 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 2 - protein, 133 aa, Mus musculis
MAQMMTLSLLSLDLALCIPWTQGSDGGGQDCCLKYSQKKIPYSIVRGYRKQEPSLGCPIPAILFLPRKHSKPELCANPEEGWVQNLMRRLDQPPAPGKQSPGCRKNRGTSKSGKKGKGSKGCKRTEQTQPSRG

SEQ ID NO: 3 - DNA, 23 bp, Homo sapiens
tggaccttctaggtcttgaaagg

SEQ ID NO: 4 - DNA, 24 bp, Homo sapiens
aggcattccaccactgctcccatt